(19) United States
(12) Patent Application Publication
(10) Pub. No.: US 2018/0080940 A1
(43) Pub. Date: Mar. 22, 2018

(54) TESTING DEVICE AND METHOD FOR PRODUCING SAME, AND TESTING KIT AND TRANSFER MEDIUM FOR PRODUCING TESTING DEVICE

(71) Applicants: Miyuki Hirata, Mie (JP); Rie Kobayashi, Kanagawa (JP)

(72) Inventors: Miyuki Hirata, Mie (JP); Rie Kobayashi, Kanagawa (JP)

(21) Appl. No.: 15/707,522

(22) Filed: Sep. 18, 2017

(30) Foreign Application Priority Data
Sep. 20, 2016 (JP) 2016-183339

(51) Int. Cl. G01N 33/58

(52) U.S. Cl. CPC G01N 33/585

(57) ABSTRACT

Provided is a testing device including: a porous flow path member in which a testing liquid containing a sample is flowed; a resin layer provided at at least one position over the flow path member; a solid-phase reagent containing an antibody reactive with the sample; and a reagent deposited portion that is water-soluble and contains a water-soluble resin and the solid-phase reagent, wherein the resin layer contains a water-insoluble resin and is provided with the solid-phase reagent and the reagent deposited portion over a surface of the resin layer facing the flow path member, and wherein the reagent deposited portion is disposed between the flow path member and the resin layer.

### CROSS-REFERENCE TO RELATED APPLICATIONS

The present application claims priority under 35 U.S.C. § 119 to Japanese Patent Application No. 2016-183339, filed Sep. 20, 2016. The contents of which are incorporated herein by reference in their entirety.

### BACKGROUND OF THE INVENTION

### Field of the Invention

The present disclosure relates to a testing device and a method for producing the same, and a testing kit and a transfer medium for producing a testing device.

### Description of the Related Art

Immunoassay, which is a method for detecting and quantifying an antigen or an antibody utilizing a reaction between the antigen and the antibody, is widely used for, for example, testing samples such as blood, DNAs, foods, and beverages.

ELISA (Enzyme Linked Immuno Solvent Assay) methods, which are representative examples of the immunoassay, are methods for quantitatively analyzing antibodies or antigens by filling a plastic container called microplate and including a plurality of wells with antibodies or antigens and reading the absorbance of the reaction products with a dedicated measuring instrument. These methods enable high-sensitivity analyses, in a manner of forming antibodies as high-density solid phases over the surfaces of the microplate within the wells and allowing the antibodies to undergo antigen-antibody reactions for some hours batch-wise. Therefore, the methods are used in, for example, ordinary laboratories and clinical laboratories of medical institutions.

Recently, there has been a rapid penetration of measuring methods intended for POCT (Point of Care Testing). As representative examples, testing devices employing measuring methods called immunochromato methods in which the principle of sandwich ELISA methods and the principle of chromatography are combined have been used.

As such a testing device, there has been proposed a device including: a sample pad serving as a liquid receiving portion for receiving a testing liquid; a conjugate pad in which the testing liquid supplied from the sample pad is allowed to undergo a reaction; and a membrane film in which the testing liquid supplied from the conjugate pad flows (see, for example, Japanese Unexamined Patent Application Publication No. 2010-256309).

### SUMMARY OF THE INVENTION

According to one aspect of the present disclosure, a testing device includes a porous flow path member in which a testing liquid containing a sample is flowed, a resin layer provided at at least one position over the flow path member, a solid-phase reagent containing an antibody reactive with the sample, and a reagent deposited portion that is water-soluble and contains a water-soluble resin and the solid-phase reagent. The resin layer contains a water-insoluble resin and is provided with the solid-phase reagent and the reagent deposited portion over a surface of the resin layer facing the flow path member. The reagent deposited portion is disposed between the flow path member and the resin layer.

### BRIEF DESCRIPTION OF THE DRAWINGS

FIG. 1 is a top view illustrating an example of a testing device of the present disclosure;

FIG. 2 is a cross-sectional view of the testing device of FIG. 1 taken along a line A-A;

FIG. 3 is a schematic cross-sectional view illustrating an example of a testing device taken at a portion at which a flow path member and a resin layer face each other;

FIG. 4A is a schematic cross-sectional view illustrating another example of a testing device taken at a portion at which a flow path member and a resin layer face each other;

FIG. 4B is a schematic cross-sectional view illustrating another example of a testing device taken at a portion at which a flow path member and a resin layer face each other;

FIG. 4C is a schematic cross-sectional view illustrating another example of a testing device taken at a portion at which a flow path member and a resin layer face each other;

FIG. 4D is a schematic cross-sectional view illustrating another example of a testing device taken at a portion at which a flow path member and a resin layer face each other;

FIG. 5A is a schematic cross-sectional view illustrating another example of a testing device taken at a portion at which a flow path member and a resin layer face each other;

FIG. 5B is a schematic cross-sectional view illustrating another example of a testing device taken at a portion at which a flow path member and a resin layer face each other;

FIG. 5C is a schematic cross-sectional view illustrating another example of a testing device taken at a portion at which a flow path member and a resin layer face each other;

FIG. 5D is a schematic cross-sectional view illustrating another example of a testing device taken at a portion at which a flow path member and a resin layer face each other;

FIG. 6 is a conceptual diagram of a conjugate pad of an existing testing device;

FIG. 7 is a conceptual diagram of a membrane of an existing testing device;

FIG. 8A is a schematic cross-sectional view illustrating an example of a transfer medium for producing a testing device of the present disclosure;

FIG. 8B is a schematic cross-sectional view illustrating another example of a transfer medium for producing a testing device of the present disclosure;

FIG. 9 is a schematic diagram illustrating an example of a testing kit of the present disclosure;

FIG. 10A is a top view illustrating an example of a testing device used in Example; and

FIG. 10B is a cross-sectional view of FIG. 10A taken along a line D-D.

### DESCRIPTION OF THE EMBODIMENTS

### (Testing Device)

In a first embodiment, a testing device of the present disclosure includes a porous flow path member in which a testing liquid containing a sample is flowed, a resin layer provided at at least one position over the flow path member, a solid-phase reagent containing an antibody reactive with the sample, and a reagent deposited portion that is water-soluble and contains a water-soluble resin and the solid-phase reagent. The resin layer contains a water-insoluble resin and is provided with the solid-phase reagent and the reagent deposited portion over a surface of the resin layer facing the flow path member. The reagent deposited portion is disposed between the flow path member and the resin layer.

The resin layer is provided at at least one position, preferably at a plurality of positions over the flow path member.

In a second embodiment, a testing device of the present disclosure includes a porous flow path member in which a testing liquid containing a sample is flowed, and a first resin layer and a second resin layer over the flow path member.

The second resin layer is provided with a labeled antibody over a surface of the second resin layer facing the flow path member.

The first resin layer contains a water-insoluble resin, and is provided with a capture antibody and a reagent deposited portion that is water-soluble and contains a water-soluble resin and the capture antibody, the capture antibody and the reagent deposited portion being provided over a surface of the first resin layer facing the flow path member.

It is preferable that the reagent deposited portion be disposed between the flow path member and the first resin layer.

It is preferable to provide a plurality of first resin layers, in order to confirm whether a sample moves in the testing device safely.

The testing device of the present disclosure is based on a finding that existing immunochromato methods need to flow a labeled antibody in an amount highly excessive relative to an antigen, so the labeled antibody that does not particularly become reactive immediately after the testing liquid is flowed arrives at a capture antibody portion at a high density and non-specifically adsorbs to the capture antibody portion due to hydrophobic interaction.

The present disclosure has an object to provide a testing device that can suppress color development due to non-specific adsorption and can obtain a judgement line that develops a color at a high density.

The present disclosure can provide a testing device that can suppress color development due to non-specific adsorption and can obtain a judgement line that develops a color at a high density.

In the testing device of the present disclosure, the reagent deposited portion that is water-soluble and is provided between the flow path member and the resin layer can ensure that non-specific adsorption produced during an initial period after the testing liquid is spread will not remain on the capture antibody portion (line), by eluting the non-specific adsorption together with the water-soluble resin. Further, gradually releasing the capture antibody dispersed in the water-soluble resin toward the membrane along with dissolution of the water-soluble resin makes it possible to adjust the density of the capture antibody and have control on an antigen-antibody reaction. The antigen-antibody reaction product quickly adsorbs to the fiber constituting the flow path member and does not flow out together with the testing liquid but remains in the membrane immediately below the capture antibody portion (line). This enables a more highly-sensitive detection.

Furthermore, the reagent deposited portion that is water-soluble, contains the water-soluble resin, and is provided between the flow path member and the resin layer has a cushioning effect of protecting the capture antibody in the capture antibody portion (line) from mechanical stresses (e.g., shocks). This provides an excellent stability during production and storage.

Moreover, combining the water-insoluble resin and the water-soluble resin at the resin layer enables the water-insoluble resin and the water-soluble resin to exert the functions more effectively than when an amphiphilic resin is used, because a clearer separation can be obtained between the function of the water-insoluble resin and the function of the water-soluble resin. That is, the water-soluble resin can suppress non-specific adsorption to the resin layer and improve intimacy between the testing liquid and the resin layer and a color developing property. The water-insoluble resin can provide an effect of improving color development on the surface of the resin layer effectively.

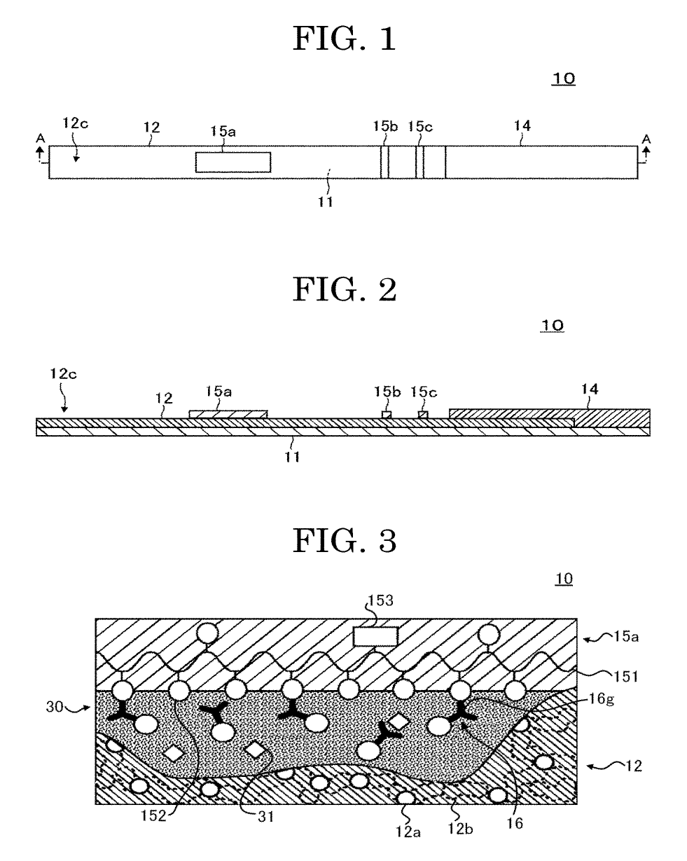

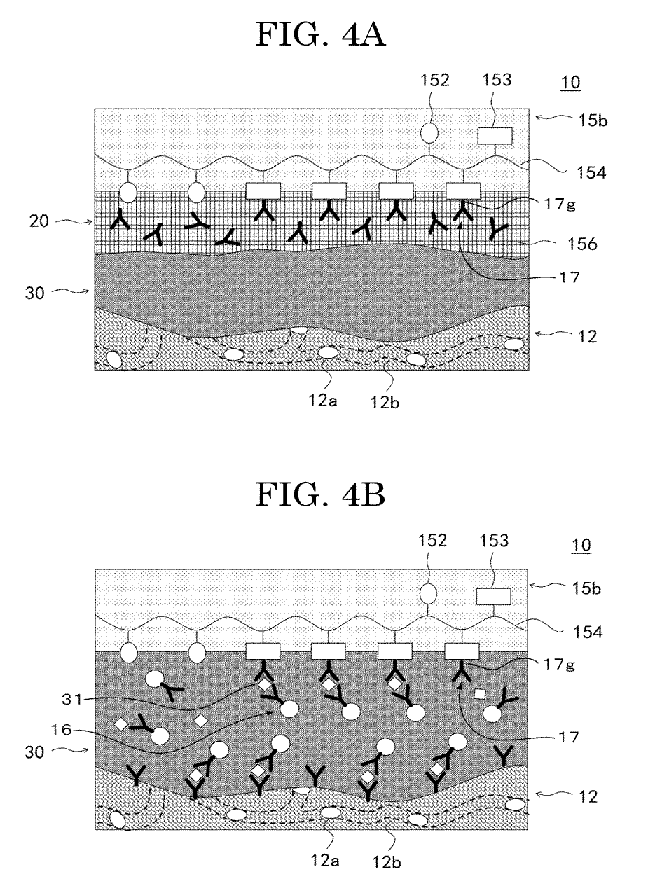

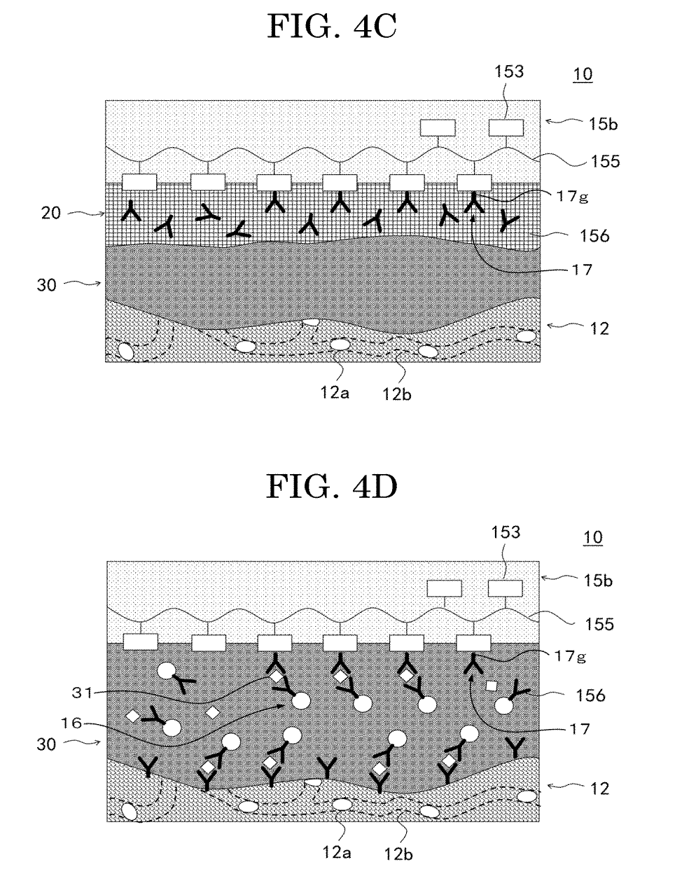

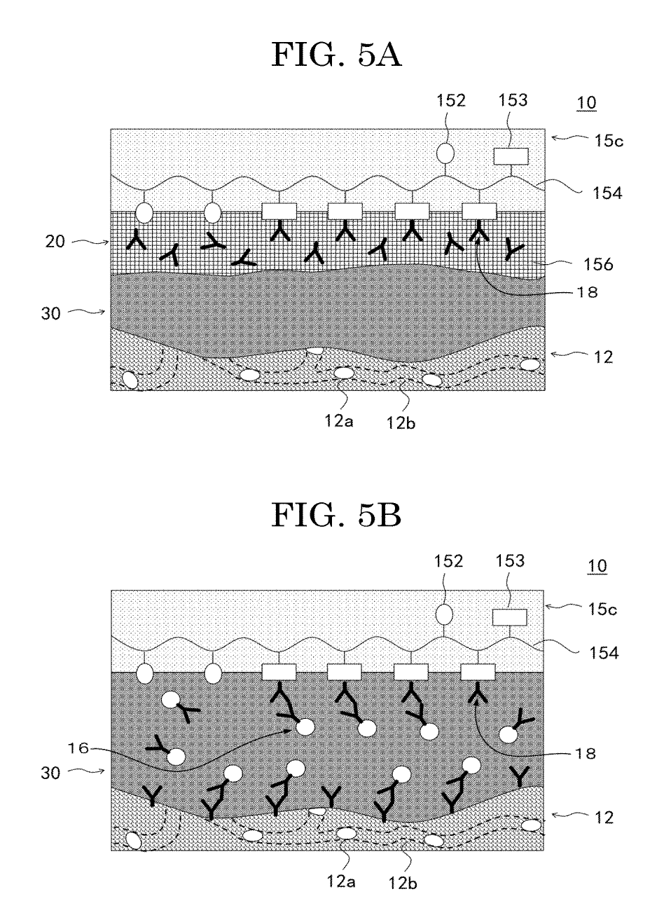

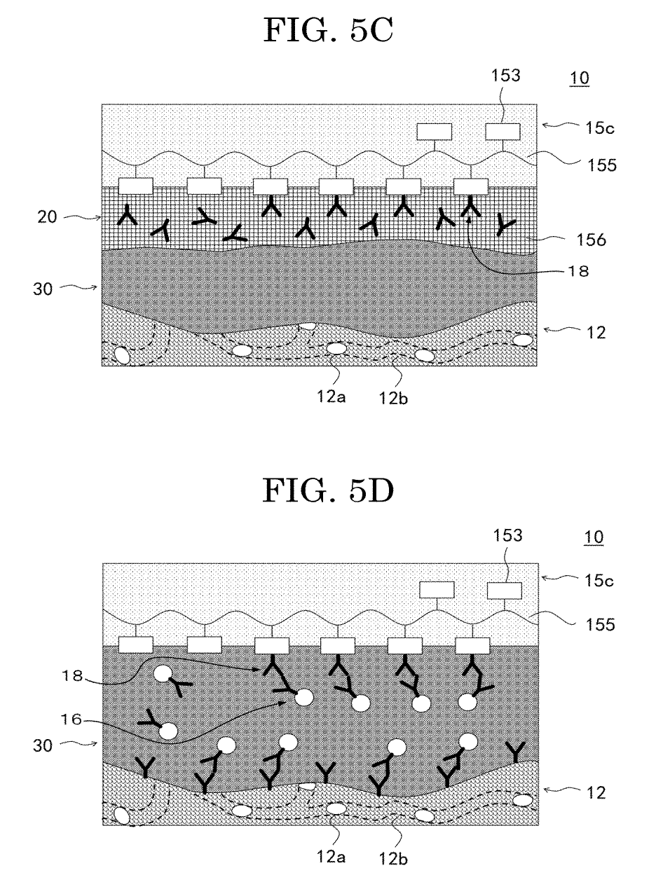

The testing device of the present disclosure will be described with reference to the drawings. FIG. 1 is a top view illustrating an example of a testing device 10 of the present disclosure. FIG. 2 is a cross-sectional view of the testing device of FIG. 1 taken along a line A-A. FIG. 3 is a schematic cross-sectional view illustrating an example of a testing device taken at a portion at which a flow path member and a resin layer face each other. FIG. 4A, FIG. 4B, FIG. 4C, FIG. 4D, FIG. 5A, FIG. 5B, FIG. 5C, and FIG. 5D are each a schematic cross-sectional view illustrating another example of a testing device taken at a portion at which a flow path member and a resin layer face each other.

As illustrated in FIG. 1 to FIG. 5D, the testing device 10 includes a porous flow path member 12 in which a flow path for flowing a hydrophilic testing liquid 30 (an example of a sample), such as blood, spinal fluid, urine, and a sample extraction liquid (e.g., a liquid containing a sample picked with a sample picking unit such as a stick) is formed, and resin layers (15a, 15b, and 15c) provided over the flow path member 12.

A labeled antibody 16, a capture antibody 17, and a capture antibody 18, which are reagents reactive with an antigen contained in the testing liquid 30, are formed as solid phases over the surfaces of the resin layers (15a, 15b, and 15c) facing the flow path member 12. This enables the intensity of interaction between the resin layers (15a, 15b, and 15c) and the reagents to be adjusted independently for each of the resin layers (15a, 15b, and 15c). This makes it easy to control release of the reagents and formation of the reagents as solid phases, even when the flow path member 12 is arbitrary selected depending on the purpose.

A case where in the testing device 10, the flow path member 12 is provided over a base material 11, and an absorbing member 14 is provided over the base material 11 and the flow path member 12 will be described. However, the present disclosure is not limited to this embodiment.

What is meant when it is said that something is provided over the flow path member 12 is that that something is provided in a manner to contact the flow path member 12 regardless of whether that something comes to which side of the flow path member when the testing device 10 is set in place. When an arbitrary resin layer of the resin layers (15a, 15b, and 15c) is to be referred to, the arbitrary resin layer will be denoted as resin layer 15. The capture antibody may be formed as a solid phase by arbitrary chemical binding such as covalent binding, hydrogen binding, and metal binding and arbitrary interaction such as adhesion, cohesion, adsorption, and van der Waals binding.

A case where the testing liquid 30 is a hydrophilic testing liquid 30 such as blood, spinal fluid, urine, and a sample extraction liquid (e.g., a liquid containing a sample picked with a sample picking unit such as a stick) will be described below.

As illustrated in FIG. 3, in the testing device 10, the resin layer 15a (second resin layer) contains an amphiphilic resin 151 containing many hydrophilic groups 152. The amphiphilic resin 151 is preferably a main component (accounting for 50% by mass or greater) of the resin layer 15a.

A hydrophilic group is a group of atoms forming a weak bond with water molecules by, for example, hydrogen binding, and has affinity with water. Amphiphilicity means that a substance has affinity with both of water and organic solvents.

The labeled antibody 16 has a hydrophilic portion 16g, by which the labeled antibody 16 is formed as a solid phase over the surface of the resin layer 15a facing the flow path member 12. Meanwhile, when the gap formed in the facing portion at which the flow path member 12 and the resin layer 15a face each other is filled with the testing liquid 30, the hydrophilic portion 16g of the labeled antibody 16 comes to have affinity with the hydrophilic testing liquid 30 to cause the labeled antibody 16 to be released from the amphiphilic resin 151. When the testing liquid contains an antigen 31, the released labeled antibody 16 and the antigen 31 react and bind with each other by an antigen-antibody reaction.

As illustrated in FIG. 4A to FIG. 4D, in the testing device 10, it is preferable that the resin layer 15b (first resin layer) be a resin layer containing a hydrophobic group 153. Specifically, the resin layer 15b contains a hydrophobic resin 155 or an amphiphilic resin 154 containing many hydrophobic groups 153. The hydrophobic resin 155 or the amphiphilic resin 154 is preferably a main component (accounting for 50% by mass or greater) of the resin layer 15b.

A hydrophobic group is a group of atoms having a poor intimacy with water or a poor affinity with water and is sparingly soluble in water or sparingly miscible with water.

It is preferable that both of the hydrophobic resin 155 and the amphiphilic resin 154 be the water-insoluble resin mentioned above. When the hydrophobic resin 155 and the amphiphilic resin 154 are the water-insoluble resin, there is an advantage that feathering of the line can be prevented.

“Water-insolubility” of the water-insoluble resin means substantial water-insolubility. Substantial water-insolubility means that a resin undergoes a mass change in an amount of 1% by mass or less when immersed in a large amount of water at 25 degrees C. for 24 hours and then sufficiently dried by a method such as vacuum drying. The reason why such a resin is substantially water-insoluble is that the mass change is attributed to mass reduction due to leaching of a by-product (e.g., a monomer component) contained in the resin into the water.

The capture antibody 17 has a hydrophobic portion 17g. The capture antibody 17 is formed as a solid phase over a surface of the resin layer 15b facing the flow path member 12, by the hydrophobic portion 17g binding with that surface by an intermolecular force.

Furthermore, a reagent deposited portion 20 that is water-soluble and contains the capture antibody 17 and a water-soluble resin is disposed between the flow path member 12 and the resin layer 15b. Therefore, when the portion between the flow path member 12 and the resin layer 15b is filled with the testing liquid 30, the water-soluble resin is eluted toward the flow path member 12 immediately below, to immobilize the capture antibody 17 to the flow path member 12. The capture antibody 17 immobilized to the flow path member 12 captures the antigen 31 that is in the state of being bound with the labeled antibody 16. Besides, when the gap formed in the facing portion at which the flow path member 12 and the resin layer 15b face each other is filled with the testing liquid 30, the capture antibody 17 captures the antigen 31 that is in the state of being bound with the labeled antibody 16. As a result, the antigen 31 and the labeled antibody 16 are immobilized and develop a color. Therefore, the resin layer 15b can be used as a test line for judging presence or absence of the antigen 31. Moreover, color development also occurs in the flow path member 12 immediately below the resin layer 15b, to make the line for judging presence or absence of the antigen 31 more highly perceivable.

As illustrated in FIG. 5A to FIG. 5D, in the testing device 10, the resin layer 15c (first resin layer) contains a hydrophobic resin 155 or an amphiphilic resin 154 containing many hydrophobic groups 153. The hydrophobic resin 155 or the amphiphilic resin 154 is preferably a main component (accounting for 50% by mass or greater) of the resin layer 15c.

It is preferable that both of the hydrophobic resin 155 and the amphiphilic resin 154 be the water-insoluble resin mentioned above. When the hydrophobic resin 155 and the amphiphilic resin 154 are the water-insoluble resin, there is an advantage that feathering of the line can be prevented.

The capture antibody 18 is formed as a solid phase over a surface of the resin layer 15c facing the flow path member 12, by a hydrophobic portion of the capture antibody 18 binding with that surface by an intermolecular force. The capture antibody 18 is not particularly limited and may be appropriately selected depending on the intended purpose, so long as the capture antibody 18 can capture the labeled antibody 16. Examples of the capture antibody 18 include an antibody that specifically binds with the labeled antibody 16.

Furthermore, a reagent deposited portion 20 that is water-soluble and contains the capture antibody 18 and a water-soluble resin is disposed between the flow path member 12 and the resin layer 15c. Therefore, when the portion between the flow path member 12 and the resin layer 15c is filled with the testing liquid 30, the water-soluble resin is eluted toward the flow path member 12 immediately below, to immobilize the capture antibody 18 to the flow path member 12. The capture antibody 18 immobilized to the flow path member 12 captures the labeled antibody 16. Besides, when the gap formed in the facing portion at which the flow path member 12 and the resin layer 15c face each other is filled with the testing liquid 30, the capture antibody 18 captures the labeled antibody 16. As a result, the labeled antibody 16 is immobilized and develops a color. Therefore, the resin layer 15c can be used as a control line for indicating that the labeled antibody 16 has arrived. Moreover, color development also occurs in the flow path member 12 immediately below the resin layer 15c, to make the line more highly perceivable.

The resin layers are preferably non-porous bodies. The non-porous body refers to a non-porous structure substantially free of voids, and a structure opposed to a porous material such as a membrane that contains voids provided for promoting absorption of a liquid. Hence, a material that contains only few cells that have been incidentally mixed in the material during a production process and that do not contribute to promotion of the liquid absorbing action is encompassed within the non-porous body.

Hitherto, test lines and control lines have been formed by directly coating a liquid in which a capture antibody is dissolved over the flow path member formed of a hydrophilic porous material. Hence, the capture antibody diffuses inside the porous material along with permeation of the liquid. However, a color developed by labeling particles such as gold colloid particles to be bound with the capture antibody present inside the porous material cannot actually be sensed due to light scattering. This means that most of the capture antibody is not used effectively.

Generally, color developing particles that can be sensed from the porous material are particles that are present at and above the depth of about 5 micrometers from the surface of the porous material. In order to immobilize the capture antibody needed for testing to the region at and above the depth of 5 micrometers, there is a need for coating the capture antibody in a large amount considering diffusion of the capture antibody in the direction of thickness. That is, the amount of the capture antibody to be coated increases in proportion to the thickness of the porous material.

In the testing device of the present disclosure, the resin layer formed of a non-porous body containing many hydrophobic groups is used for immobilization of the capture antibody. Therefore, the capture antibody is immobilized to only the surface of the resin layer without entering the inside of the resin layer. A color is developed when labeling particles bind with the capture antibody immobilized to the surface of the resin layer. The color can be sensed through the resin layer formed of the non-porous body that does not scatter light. This significantly improves the efficiency of utilization of the color developed by the labeling particles. Because there are no wasteful color developing particles in the direction of thickness, there is an advantage that the amount of the capture antibody coated can be significantly suppressed. For example, when it is assumed that the thickness of the flow path member formed of the hydrophilic porous material is 100 micrometers and color development from a region at and above the depth of 5 micrometers from the surface of the flow path member can only be utilized, the amount of the capture antibody coated used for obtaining color development of the same intensity can be reduced to 1/20. That is, the resin layer formed of a non-porous body containing many hydrophobic groups is used for immobilization of the capture antibody. Therefore, the efficiency of utilization of the color developed by the labeling particles can be improved significantly, and the amount of the capture antibody coated can be reduced from the amount used in existing devices because there are no wasteful labeling particles in the direction of thickness.

Here, the testing device 10 configured to test presence or absence of the antigen 31 in the testing liquid 30 is described. However, the testing device of the present disclosure is not limited to a testing device utilizing an antigen-antibody reaction. For example, the testing device may be configured to test a specific component in the testing liquid 30 by using, as a reagent, a reagent that changes hues upon a structural change.

Each member constituting the testing device 10 will be described in detail below.

### <Base Material>

The base material 11 is not particularly limited and may be appropriately selected depending on the intended purpose. Examples of the base material include organic, inorganic, and metallic base materials.

The base material 11 is not particularly limited and may be appropriately selected depending on the intended purpose. However, it is preferable that at least one surface of the base material 11 be coated with a hydrophobic resin.

When the testing device 10 is used as a sensor chip, it is preferable to use a light-weight, flexible, and inexpensive synthetic resin as the base material 11.

In the present disclosure, it is optional to select a base material 11 having a high durability such as a plastic sheet. This improves the durability of the testing device 10 as a result.

A constituent material of the base material 11 is not particularly limited and may be appropriately selected depending on the intended purpose. Examples of the constituent material of the base material 11 include polyvinyl chlorides, polyethylene terephthalates, polypropylenes, polystyrenes, polyvinyl acetates, polycarbonates, polyacetals, modified polyphenyl ethers, polybutylene phthalates, and ABS resins. One of these materials may be used alone or two or more of these materials may be used in combination. Among these materials, polyethylene terephthalates are preferable because polyethylene terephthalates are low-price and highly versatile.

The shape of the base material 11 is not particularly limited and may be appropriately selected depending on the intended purpose. However, a sheet shape is preferable.

The average thickness of the base material 11 is not particularly limited, may be appropriately selected depending on the intended purpose, and is preferably 0.01 mm or greater but 0.5 mm or less. When the average thickness of the base material 11 is 0.01 mm or greater, the base material 11 has an adequate strength as a base material. When the average thickness of the base material 11 is 0.5 mm or less, the base material has a good flexibility and is suitable as a sensor.

The average thickness may be an average of thicknesses measured with a micrometer (MDH-25M available from Mitutoyo Corporation) at a total of 15 positions of a measuring target, namely 5 positions in the longer direction×3 positions in the width direction that are selected at approximately equal intervals. The thickness may be a length of a target in a direction perpendicular to a contact plane at which the base material 11 and the flow path member 12 contact each other.

### <Flow Path Member>

The flow path member 12 is not particularly limited and may be appropriately selected depending on the intended purpose so long as the flow path member is a member through which the testing liquid 30 can be flowed. Examples of the flow path member 12 include a hydrophilic porous material.

The flow path member 12 formed of the hydrophilic porous material contains voids (12a and 12b), and a flow path is formed when the testing liquid 30 flows through the voids (12a and 12b).

In FIG. 3 to FIG. 5D, the void 12a is a void formed in the cross-sections, and the void 12b is a void present more backward in the cross-sections. It is preferable that cells be present in the hydrophilic porous material and that the cells be linked together to form a continuous cell.

The continuous cell is distinguished from independent cells that are not linked together. The continuous cell has a function of sucking in a liquid by a capillary action or letting a gas pass through the continuous cell because the continuous cell has small holes in the walls between the cells. The flow path member 12 needs no external actuating device such as a pump because the flow path member 12 is configured to deliver the testing liquid 30 by utilizing a capillary action through the voids (12a and 12b).

The hydrophilic porous material is not particularly limited and may be appropriately selected depending on the intended purpose. However, a material having hydrophilicity and a high voidage is preferable.

The hydrophilic porous material refers to a porous material that is easily permeable by an aqueous solution. The hydrophilic porous material is referred to as being easily permeable when in a water permeability evaluation test in which 0.01 mL of pure water is dropped onto a surface of a plate-shaped test piece of the hydrophilic porous material dried at 120 degrees C. for 1 hour, the whole of 0.01 mL of the pure water permeates the test piece in 10 minutes.

The voidage of the hydrophilic porous material is not particularly limited, may be appropriately selected depending on the intended purpose, and is preferably 40% or higher but 90% or lower and more preferably 65% or higher but 80% or lower. When the voidage of the hydrophilic porous material is 90% or lower, the flow path member has a good strength. When the voidage of the hydrophilic porous material is 40% or higher, permeability of the testing liquid is good.

The voidage of the hydrophilic porous material can be calculated according to a calculation formula 1 below based on a basis weight (g/m²) and a thickness (micrometer) of the hydrophilic porous material and the specific gravity of the component of the hydrophilic porous material. [Calculation formula 1]

Voidage (%)={1−[basis weight (g/m²)/thickness (micrometer)/specific gravity of the component]}×100

The hydrophilic porous material is not particularly limited and may be appropriately selected depending on the intended purpose. Examples of the hydrophilic porous material include filter paper such as membrane film, plain paper, high-quality paper, watercolor paper, Kent paper, synthetic paper, synthetic resin film, special-purpose paper with a coat layer, fabric, fiber product, film, inorganic substrate, and glass. One of these hydrophilic porous materials may be used alone or two or more of these hydrophilic porous materials may be used in combination. Among these hydrophilic porous materials, filter paper such as membrane film and fabric are preferable, and filter paper such as membrane film is more preferable because filter paper has a high voidage and a good hydrophilicity.

Examples of the fabric include artificial fiber such as rayon, bemberg, acetate, nylon, polyester, and vinylon, natural fiber such as cotton and silk, blended fabric of these fibers, or non-woven fabric of these fibers.

The shape of the hydrophilic porous material is not particularly limited and may be appropriately selected depending on the intended purpose. However, a sheet shape is preferable.

The average thickness of the hydrophilic porous material is not particularly limited, may be appropriately selected depending on the intended purpose, and is preferably 0.01 mm or greater but 0.3 mm or less. When the average thickness of the hydrophilic porous material is 0.01 mm or greater, the flow path member has a good strength. When the average thickness of the hydrophilic porous material is 0.3 mm or less, the amount of the testing liquid needed can be optimized.

### <Resin Layer>

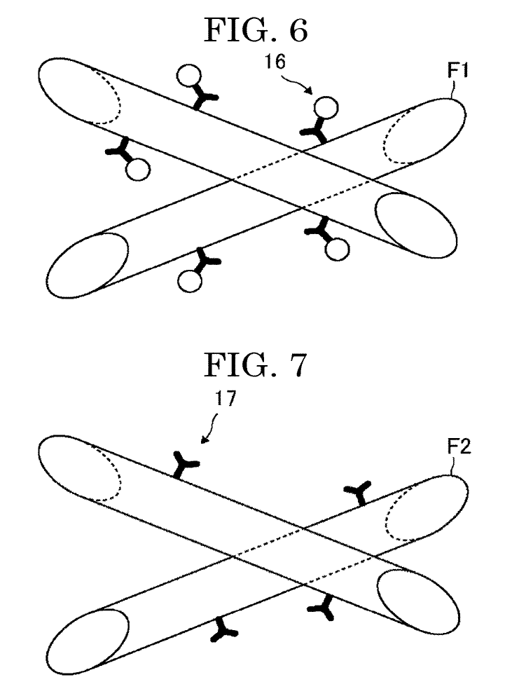

The function of the resin layer 15 will be described based on comparison with an existing testing device illustrated in FIG. 6 and FIG. 7.

FIG. 6 is a conceptual diagram of a conjugate pad of an existing testing device. FIG. 7 is a conceptual diagram of a membrane of an existing testing device.

In the existing testing device, when the conjugate pad has an extremely high hydrophilicity, a testing liquid tends to stay within the conjugate pad and does not easily flow into the membrane. Conversely, when the conjugate pad has an extremely high hydrophobicity, the testing liquid smoothly flows into the membrane, but a long time is needed for testing or the testing liquid is needed in a large amount because the conjugate pad has a poor absorbency for absorbing the testing liquid from the sample pad. Hence, fiber F1 usable for the conjugate pad is limited. Furthermore, in the existing testing device, the labeled antibody 16 is formed as a solid phase on the fiber F1 constituting the conjugate pad (see FIG. 6). The labeled antibody 16 can be released from the conjugate pad only when the labeled antibody 16 has a weak force of binding with the fiber F1. That is, as a matter of design, the existing testing device is limited in the fiber F1 and the labeled antibody 16 that can be used.

Likewise, in the existing testing device, the capture antibody 17 is formed as a solid phase on fiber F2 constituting the membrane (see FIG. 7). Hence, the capture antibody 17 can be immobilized to the membrane only when the capture antibody 17 has a strong force of binding with the fiber F2. That is, as a matter of design, the existing testing device is limited in the fiber F2 and the capture antibody 17 that can be used.

In the testing device 10 of the present disclosure, the reagents such as the labeled antibody 16, the capture antibody 17, and the capture antibody 18 are formed as solid phases over the resin layers 15 (15a, 15b, and 15c). Hence, release or immobilization of the capture antibodies can be controlled based on the intensity of interaction between the resin layers 15 and the capture antibodies and affinity between the resin layers 15 and the testing liquid 30.

Particularly, the resin layers (15b and 15c) are provided with a predetermined amount of the reagent deposited portion 20 that is water-soluble between the resin layers 15b and 15c and the flow path member 12. This makes it possible to gradually release the antibody dispersed in the water-soluble resin toward the membrane along with dissolution of the water-soluble resin and to make an antigen-antibody reaction occur with the density of the capture antibody controlled. The antigen-antibody reaction product quickly adsorbs to the fiber constituting the flow path member 12 and does not flow out together with the testing liquid 30 but remains in the membrane immediately below the capture antibody portion (judgment line). This enables a more highly-sensitive detection.

As the method for adjusting the intensity of interaction between the resin layers 15 and the capture antibodies and affinity between the resin layers 15 and the testing liquid 30, for example, there is a method of changing the kinds of the resins to constitute the resin layers 15 or the composition ratios of the resins in a manner to match the corresponding capture antibodies. For example, the higher the hydrophobic percentage in the resin constituting the resin layer 15, the easier it is to immobilize a capture antibody containing a hydrophobic group to the resin layer 15 based on hydrophobic interaction.

The hydrophobic interaction refers to a cause (driving force) of a change occurring in water that hydrophobic molecules or hydrophobic groups immiscible with water aggregate with each other. To be more specific, when hydrophobic molecules or molecules having hydrophobic groups are put in water, in many cases, these molecules not only simply do not dissolve but come into a state of the hydrophobic molecules and the hydrophobic groups contacting each other to reduce the area of contact with water molecules as much as possible. The hydrophobic interaction refers to a consequent phenomenon that the hydrophobic molecular species attract each other and seem to have a binding force acting between the molecules.

When the hydrophilic percentage is high in the resin constituting the resin layer 15, the resin layer 15 has a strong interaction with a hydrophilic capture antibody. However, it is estimated that when the bonding portion between the resin layer and the capture antibody contacts the hydrophilic testing liquid 30, the reagent comes to have affinity with the testing liquid 30 and is easily released into the testing liquid 30.

The resin constituting the resin layer 15 is preferably a water-insoluble resin. A water-insoluble resin, when used in the resin layer 15, can be kept from being dissolved in the testing liquid 30 and hence clogging the flow path or smudging the control line or the test line.

The water-insoluble resin is not particularly limited and may be appropriately selected depending on the intended purpose. Examples of the water-insoluble resin include resins constituting the resin layer 15b and the resin layer 15c described below.

The amphiphilic resin constituting the resin layer 15a is not particularly limited and may be appropriately selected depending on the intended purpose. Examples of the amphiphilic resin include polyvinyl alcohols, polyvinylacetal resins, polyacrylic acids, polyacrylic acid-acrylonitrile copolymers, vinyl acetate-acrylic acid ester copolymers, acrylic acid-acrylic acid ester copolymers, styrene-acrylic acid copolymers, styrene-methacrylic acid copolymers, styrene-methacrylic acid-acrylic acid ester copolymers, styrene-α-methylstyrene-acrylic acid copolymers, styrene-α-methylstyrene-acrylic acid-acrylic acid ester copolymers, styrene-maleic acid copolymers, styrene-maleic anhydride copolymers, vinyl naphthalene-acrylic acid copolymers, vinyl naphthalene-maleic acid copolymers, vinyl acetate-maleic acid ester copolymers, vinyl acetate-crotonic acid copolymers, vinyl acetate-acrylic acid copolymers, and salts of these amphiphilic resins. One of these amphiphilic resins may be used alone or two or more of these amphiphilic resins may be used in combination.

Among these amphiphilic resins, copolymers of hydrophobic functional group-containing monomers and hydrophilic functional group-containing monomers and polymers formed of monomers containing both of hydrophobic functional groups and hydrophilic functional groups are preferable.

As the form of the copolymers, any of random copolymers, block copolymers, alternating copolymers, and graft copolymers may be used.

Examples of the hydrophobic resin constituting the resin layer 15b and the resin layer 15c include: polystyrene-based resins such as polystyrenes and acrylonitrile-butadiene-styrene copolymers; polyolefin-based resins or cyclic polyolefin-based resins such as polypropylene resins, polyethylene resins, and ethylene-propylene copolymers; polycarbonate resins, polyethylene terephthalate resins, methacrylic-based resins such as polymethyl methacrylate resins; fluororesins such as polytetrafluoroethylene; and acrylic-based resins such as polyacrylonitrile, cellulose-based resins such as propionate resins, vinyl chloride resins, polybutylene terephthalate resins, polyarylate resins, polysulfone resins, polyether sulfone resins, polyether ether ketone resins, polyether imide resins, and polymethylpentene resins. One of these hydrophobic resins may be used alone or two or more of these hydrophobic resins may be used in combination.

Examples of compounds that may constitute the resin layer 15b and the resin layer 15c other than the hydrophobic resins include: natural waxes such as beeswax, carnauba wax, cetaceum, Japan wax, candelilla wax, rice bran wax, and montan wax; synthetic waxes such as paraffin wax, microcrystalline wax, oxidized wax, ozokerite, ceresin, ester wax, polyethylene wax, and polyethylene oxide wax; higher fatty acids such as margaric acid, lauric acid, myristic acid, palmitic acid, stearic acid, furoic acid, and behenic acid; higher alcohols such as stearic alcohol and behenyl alcohol; esters such as fatty acid ester of sorbitan; and amides such as stearin amide and oleic amide. One of these compounds may be used alone or two or more of these compounds may be used in combination.

Among the compounds that may constitute the resin layer 15b and the resin layer 15c, polystyrene resins, polyolefin resins, carnauba wax, and polyethylene wax are preferable because these compounds have a strong hydrophobic interaction.

The resin layers (15a, 15b, and 15c) may be formed of the same resin. In this case, it is preferable that the resin constituting the resin layer 15a be higher in hydrophilicity than the resins constituting the resin layers (15b and 15c). Note that the same resin can be said to have a higher hydrophilicity when the percentage of hydrophilic groups is higher, without the need for measuring hydrophilicity.

The labeled antibody 16 to be formed as a solid phase over the resin layer 15a is not particularly limited and may be appropriately selected depending on the intended purpose, so long as the labeled antibody 16 has a hydrophilic portion and is reactive with the antigen 31. Examples of the labeled antibody 16 include gold colloid-labeled antibodies such as gold colloid-labeled anti-human IgG, labeled antibodies against various allergens, and particles for labeling other antibodies.

The particles for labeling other antibodies are not particularly limited to gold colloid and may be appropriately selected depending on the intended purpose. Examples of such particles include metal colloids other than gold colloid, enzymatic labeling particles containing an enzyme, coloring particles containing a pigment, fluorescent particles containing a fluorescent substance, and magnetic body encapsulating particles containing a magnetic body. One of these kinds of particles may be used alone or two or more of these kinds of particles may be used in combination.

Examples of the antibody include monoclonal antibody, polyclonal antibody, chimeric antibody, Fab antibody, and (Fab)₂ antibody.

The capture antibody 17 to be formed as a solid phase over the resin layer 15b is not particularly limited and may be appropriately selected depending on the intended purpose, so long as the capture antibody 17 has a hydrophobic portion and is reactive with the antigen 31. Examples of the capture antibody 17 include antibodies such as anti-human IgG and antibodies against various allergens.

Examples of the antibody include monoclonal antibody, polyclonal antibody, chimeric antibody, Fab antibody, and (Fab)₂ antibody.

The capture antibody 18 to be formed as a solid phase over the resin layer 15c is not particularly limited and may be appropriately selected depending on the intended purpose, so long as the capture antibody 18 has a hydrophobic group and is reactive with the labeled antibody 16. Examples of the capture antibody 18 include antibodies such as human IgG against the labeled antibody 16 and antibodies raised as examples above.

The method for forming the reagents such as the labeled antibody 16 and the capture antibodies (17 and 18) as solid phases over the resin layers 15 is not particularly limited and may be appropriately selected depending on the intended purpose. Examples of the method include a method of coating a solution containing a reagent such as a capture antibody over the resin layer 15 and then drying up the solution by fast drying, and a method of coating a solution containing a reagent over the resin layer 15, leaving the coated resin layer standing still (for incubation) in a high humidity environment so as not for the coating liquid to dry, cleaning away any other components than the antibody such as an inorganic salt with, for example, distilled water, and then drying the antibody.

The method for treating a reagent such as the capture antibody as a coating liquid is not particularly limited and may be appropriately selected depending on the intended purpose. However, typically, it is preferable to dilute the reagent with a buffer solution (buffering agent) called buffer.

The buffer solution is not particularly limited so long as the buffer solution is a buffer that does not inhibit antigen-antibody reaction of the capture antibody. Examples of the buffer solution include buffer solutions commonly used for diluting antibodies. The buffer solution may be, for example, phosphate buffered saline (PBS), tris buffered saline (TBS), trishydroxymethylaminomethane (Tris)-HCl, and good buffer. One of these buffer solutions may be used alone or two or more of these buffer solutions may be used in combination. Among these buffer solutions, phosphate buffered saline (PBS) and good buffer are preferable. The pH of the phosphate buffered saline (PBS) is preferably from 4 through 10 and more preferably from 6 through 8. The composition of the PBS is not particularly limited and there are various compositions of PBS. As an example of the composition of the PBS, there is a composition in which NaCl is 8.0 g/L, KCl is 0.2 g/L, NaH₂PO₄ is 1.44 g/L, and KH₂PO₄ is 0.24 g/L. There are also compositions free of potassium and compositions containing calcium and magnesium.

Examples of the good buffer include 2-(N-monopholino)ethanesulfonic acid (MES) and N-2-hydroxyethylpiperazine-2-sulfonic acid (HEPES).

The inorganic salt refers to a salt and an ion that can be measured by an ion chromatography method. Examples of the inorganic salt include: cations such as a sodium ion, a potassium ion, an ammonium ion, a magnesium ion, and a calcium ion; anions such as chlorine ion, a PO₄ ion, and SO₄ ion; and salts of these cations and anions.

In the present disclosure, it is preferable that the resin layer 15 be secured over the flow path member 12.

The method for securing the resin layer 15 over the flow path member 12 is not particularly limited and may be appropriately selected depending on the intended purpose, so long as the method secures the resin layer 15 in a manner that the reagent and the testing liquid 30 can contact each other during testing. Examples of the method include a method of thermally transferring the resin to constitute the resin layer onto the flow path member 12 with, for example, a thermal transfer printer, a method of transferring the resin to constitute the resin layer with a pressure applied with, for example, a dot impact printer, and a method of pasting the resin to constitute the resin layer over the flow path member 12 with, for example, a tape, an adhesive, or a tackifier.

### <Reagent Deposited Portion>

The reagent deposited portion 20 is disposed between the flow path member and the resin layers (15b and 15c), and is water-soluble.

Water-solubility has an opposite meaning to water-insolubility. The water-insolubility is as described above.

The reagent deposited portion 20 contains a water-soluble resin and a solid-phase reagent.

The water-soluble resin is not particularly limited and may be appropriately selected depending on the intended purpose. Examples of the water-soluble resin include poly(ethylene oxide), polyethylene glycol, polyvinyl alcohol, polyvinyl pyrrolidone, polyacrylic acid, and acrylic acid-based copolymers. One of these water-soluble resins may be used alone or two or more of these water-soluble resins may be used in combination.

Examples of the solid-phase reagent include the antibodies raised as examples in the description of the capture antibody 17.

It is preferable that the solid-phase reagent be dispersed in the water-soluble resin. It is also preferable that the solid-phase reagent account for 50% by mass or greater, more preferably 75% by mass or greater of the reagent deposited portion 20.

The average thickness of the reagent deposited portion 20 is preferably 1 micrometer or greater but 20 micrometers or less and more preferably 2 micrometers or greater but 10 micrometers or less. When the average thickness of the reagent deposited portion 20 is 20 micrometers or less, the water-soluble resin is sufficiently eluted when the testing liquid comes flowing, to enable the reagent dispersed in the water-soluble resin and the solid-phase reagent present over the surface of the water-insoluble resin to contribute to the reaction. When the average thickness of the reagent deposited portion 20 is 1 micrometer or greater, the reagent deposited portion can exert a sufficient effect and can also function as a protective layer.

### <Absorbing Member>

The absorbing member 14 is not particularly limited so long as the absorbing member 14 is a member configured to absorb water, and may be appropriately selected from known materials.

Examples of the absorbing member 14 include fiber such as paper and cloth, polymer compounds containing a carboxyl group or a salt of a carboxyl group, partially cross-linked bodies of polymer compounds containing a carboxyl group or a salt of a carboxyl group, and partially cross-linked bodies of polysaccharides. One of these absorbing members may be used alone or two or more of these absorbing members may be used in combination.

### <Other Members>

The other members are not particularly limited and may be appropriately selected depending on the intended purpose. Examples of the other members include a protective member, a labeled antibody support pad, and a sample dropping pad.

The protective member is a member intended for preventing contamination of a hand when the hand touches the flow path member.

The protective member is not particularly limited and may be appropriately selected depending on the intended purpose. Examples of the protective member include a housing configured to cover the testing device on the whole and a film provided over the flow path member.

When providing the protective member, it is preferable that an opening be provided in the protective member at a position to be above the dropping portion of the flow path member 12. It is preferable that an opening be provided in the protective member in order to release pressure in the flow path.

As described above, the resin layers 15 can be provided over the flow path member 12 by various methods. As an example, a case of using a thermal transfer method will be described. A transfer medium for producing a testing device of the present disclosure used in the thermal transfer method and a method for producing a testing device of the present disclosure will be described below.

### (Transfer Medium for Producing Testing Device)

In a first embodiment, a transfer medium for producing a testing device of the present disclosure includes a support, a release layer provided over the support, and a solid-phase reagent layer provided over the release layer, and further includes other members as needed.

A reagent reactive with a sample and a reagent deposited portion are provided over a surface of the solid-phase reagent layer.

In a second embodiment, a transfer medium for producing a testing device of the present disclosure includes a support and a release and solid-phase reagent layer provided over the support, and further includes other members as needed.

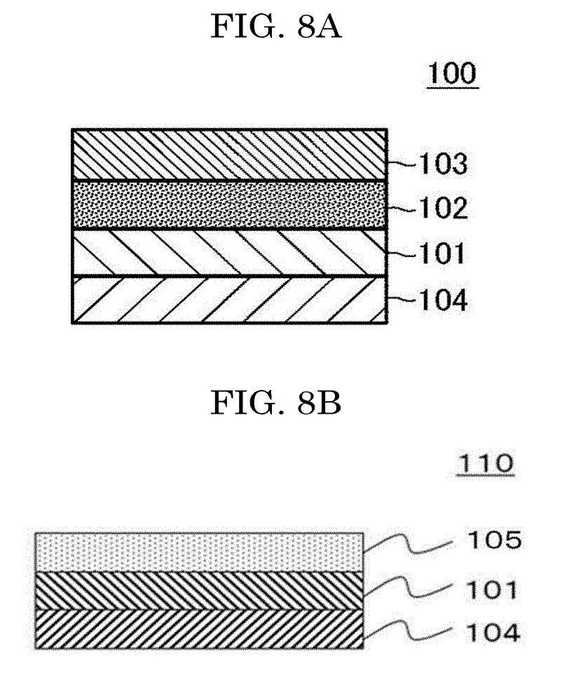

A reagent reactive with a sample and a reagent deposited portion are provided over a surface of the release and solid-phase reagent layer. The transfer medium for producing a testing device used for providing a resin layer over a flow path member will be described with reference to the drawings. FIG. 8A and FIG. 8B are schematic cross-sectional views illustrating examples of transfer media for producing a testing device of the present disclosure.

When employing the thermal transfer method, it is possible to use a transfer medium 100 for producing a testing device to which a capture antibody is previously attached uniformly. This can suppress variation in the concentration of the capture antibody (17 or 18) along a test line or a control line. When coating and locating a capture antibody by an existing method, there is a need for diluting the capture antibody with a solvent until the capture antibody has a viscosity of a coatable level (e.g., a viscosity dischargeable by an inkjet printer). On the other hand, when locating a capture antibody by thermal transfer, use of a transfer medium for producing a testing device to which a capture antibody is previously attached at a high concentration enables location of the capture antibody over a flow path at a high concentration.

As illustrated in FIG. 8A, the transfer medium 100 for producing a testing device includes a support 101, a release layer 102 provided over the support 101, and a solid-phase reagent layer 103 provided over the release layer 102. A reagent reactive with a sample and a reagent deposited portion 20 that is water-soluble are formed as solid phases over a surface of the solid-phase reagent layer 103. The transfer medium 100 for producing a testing device further includes other layers such as a back layer 104 as needed.

As represented by a transfer medium 110 for producing a testing device of FIG. 8B, a release layer 102 and a solid-phase reagent layer 103 may be provided in the form of a double-functioning release and solid-phase reagent layer 105.

### <Support>

The support 101 may be of any shape, any structure, any size, any material, etc. that are not particularly limited and may be appropriately selected depending on the intended purpose.

The structure of the support may be a single-layer structure or a laminated structure.

The size of the support may be appropriately selected depending on, for example, the size of the testing device 10.

The material of the support 101 is not particularly limited and may be appropriately selected depending on the intended purpose. Examples of the material of the support 101 include polyesters such as polyethylene terephthalate (PET) and polyethylene naphthalate (PEN), polycarbonates, polyimide resins (PI), polyamides, polyethylenes, polypropylenes, polyvinyl chlorides, polyvinylidene chlorides, polystyrenes, styrene-acrylonitrile copolymers, and cellulose acetates. One of these materials may be used alone or two or more of these materials may be used in combination. Among these materials, polyethylene terephthalate (PET) and polyethylene naphthalate (PEN) are particularly preferable.

It is preferable to apply a surface activation treatment to the surface of the support 101 in order to improve close adhesiveness with the layer to be provided over the support 101. Examples of the surface activation treatment include glow discharge treatment and corona discharge treatment.

The support 101 may be kept even after the solid-phase reagent layer 103 is transferred onto a flow path member 12, or the support 101, etc. may be peeled and removed by means of the release layer 102 after the solid-phase reagent layer 103 is transferred. When the release and solid-phase reagent layer 105 is used, the release and solid-phase reagent layer 105 may be completely transferred onto the flow path member 12, or a portion of the release and solid-phase reagent layer 105 including the surface over which the antibody is formed as a solid phase may be transferred but the release and solid-phase reagent layer 105 may be partially left over the support 101 side.

The support 101 is not particularly limited and may be an appropriately synthesized product or a commercially available product.

The average thickness of the support 101 is not particularly limited, may be appropriately selected depending on the intended purpose, and is preferably 3 micrometers or greater but 50 micrometers or less.

### <Release Layer>

The release layer 102 has a function of improving releasability between the support 101 and the solid-phase reagent layer 103 during transfer. The release layer 102 has a function of thermally melting and becoming a low-viscosity liquid when heated with a heating/pressurizing unit such as a thermal head, and making it easier for the solid-phase reagent layer 103 to be separated at about the interface between the heated portion and the non-heated portion.

The release layer 102 contains a wax and a binder resin and further contains other components as needed.

The wax is not particularly limited and may be appropriately selected depending on the intended purpose. Examples of the wax include: natural waxes such as beeswax, carnauba wax, cetaceum, Japan wax, candelilla wax, rice bran wax, and montan wax; synthetic waxes such as paraffin wax, microcrystalline wax, oxidized wax, ozokerite, ceresin, ester wax, polyethylene wax, and polyethylene oxide wax; higher fatty acids such as margaric acid, lauric acid, myristic acid, palmitic acid, stearic acid, furoic acid, and behenic acid; higher alcohols such as stearic alcohol and behenyl alcohol; esters such as fatty acid ester of sorbitan; and amides such as stearin amide and oleic amide. One of these waxes may be used alone or two or more of these waxes may be used in combination. Among these waxes, carnauba wax and polyethylene wax are preferable because these waxes are excellent in releasability.

The binder resin is not particularly limited and may be appropriately selected depending on the intended purpose. Examples of the binder resin include ethylene-vinyl acetate copolymers, partially saponified ethylene-vinyl acetate copolymers, ethylene-vinyl alcohol copolymers, ethylene-sodium methacrylate copolymers, polyamides, polyesters, polyurethanes, polyvinyl alcohols, methylcellulose, carboxymethylcellulose, starch, polyacrylic acid, isobutylene-maleic acid copolymers, styrene-maleic acid copolymers, polyacrylamides, polyvinylacetals, polyvinyl chlorides, polyvinylidene chlorides, isoprene rubbers, styrene-butadiene copolymers, ethylene-propylene copolymers, butyl rubbers, and acrylonitrile-butadiene copolymers. One of these binder resins may be used alone or two or more of these binder resins may be used in combination.

The method for forming the release layer 102 is not particularly limited and may be appropriately selected depending on the intended purpose. Examples of the method include a hot melt coating method, and a method of coating a coating liquid obtained by dispersing the wax and the binder resin in a solvent.

The average thickness of the release layer 102 is not particularly limited, may be appropriately selected depending on the intended purpose, and is preferably 0.5 micrometers or greater but 50 micrometers or less.

The amount of the release layer 102 to be attached is not particularly limited, may be appropriately selected depending on the intended purpose, and is preferably 0.5 g/m² or greater but 50 g/m² or less.

### <Solid-Phase Reagent Layer>

The solid-phase reagent layer 103 needs to contain a resin that constitutes a resin layer 15 of the testing device 10. The material of the solid-phase reagent layer 103 is not particularly limited and may be appropriately selected depending on the intended purpose.

The method for forming the solid-phase reagent layer 103 is not particularly limited and may be appropriately selected depending on the intended purpose. Examples of the method include a hot melt coating method and a method of coating a solid-phase reagent layer coating liquid obtained by dispersing the resin that constitutes the resin layer 15 in a solvent over the support 101 or the release layer 102 by a common coating method such as a gravure coater, a wire bar coater, and a roll coater, and drying the coated liquid.

The average thickness of the solid-phase reagent layer 103 is not particularly limited, may be appropriately selected depending on the intended purpose, and is preferably 200 nm or greater but 50 micrometers or less. When the average thickness of the solid-phase reagent layer 103 is 200 nm or greater, the resin layer has an improved durability and can be prevented from being damaged by, for example, friction and impact. When the average thickness of the solid-phase reagent layer 103 is 50 micrometers or less, heat from a thermal head can be uniformly conducted to the solid-phase reagent layer 103, resulting in a good definition.

The amount of a reagent coating liquid attached over the solid-phase reagent layer 103 is not particularly limited, may be appropriately selected depending on the intended purpose, and is preferably 0.2 g/m² or greater but 50 g/m² or less. When the amount of the reagent coating liquid attached is 0.2 g/m² or greater, the coating amount is appropriate and no deficiency is created in the resin layer. When the amount of the reagent coating liquid attached is 50 g/m² or less, a drying time is appropriate and no unevenness is formed in the resin layer.

### <Release and Solid-Phase Reagent Layer>

The release and solid-phase reagent layer 105 has functions of both of a release layer and a solid-phase reagent layer. The release and solid-phase reagent layer 105 can improve releasability between the support 101 and the solid-phase reagent layer 103 during transfer. Further, because the resin that constitutes the resin layer 15 of the testing device 10 is contained in the release and solid-phase reagent layer 105, a reagent such as the capture antibody 17 or the capture antibody 18 can be formed as a solid phase over the release and solid-phase reagent layer 105.

When the release and solid-phase reagent layer 105 is heated with a heating/pressurizing unit such as a thermal head, a surface of the release and solid-phase reagent layer 105 contacting the support 101 thermally melts and becomes a low-viscosity liquid (heated portion), whereas a surface of the release and solid-phase reagent layer 105 provided with the reagent as a solid phase becomes a solid state or a state close to the solid state (non-heated portion). Therefore, the release and solid-phase reagent layer 105 has a function of facilitating separation at about the interface between the heated portion and the non-heated portion.

The release and solid-phase reagent layer 105 contains a wax and a binder resin, and further contains other components as needed.

The wax is not particularly limited and may be appropriately selected depending on the intended purpose. Examples of the wax include the same waxes as raised as examples for the release layer 102. One of these waxes may be used alone or two or more of these waxes may be used in combination. Among these waxes, carnauba wax and polyethylene wax are preferable because these waxes are excellent in releasability and ability (hydrophobicity) to immobilize a capture antibody.

The binder resin is not particularly limited and may be appropriately selected depending on the intended purpose. Examples of the binder resin include the same binder resins as raised as examples for the release layer 102. One of these binder resins may be used alone or two or more of these binder resins may be used in combination.

The method for forming the release and solid-phase reagent layer 105 is not particularly limited and may be appropriately selected depending on the intended purpose. Examples of the method include a hot melt coating method, and a method of coating a coating liquid obtained by dispersing the wax and the binder resin in a solvent.

The average thickness of the release and solid-phase reagent layer 105 is not particularly limited, may be appropriately selected depending on the intended purpose, and is preferably 0.5 micrometers or greater but 50 micrometers or less. When the average thickness of the release and solid-phase reagent layer 105 is 0.5 micrometers or greater, the release and solid-phase reagent layer 105 (resin layer 15) has an improved durability and the resin layer can be prevented from being damaged by, for example, friction and impact. When the average thickness of the release and solid-phase reagent layer 105 is 50 micrometers or less, heat from a thermal head can be uniformly conducted to the release and solid-phase reagent layer 105, resulting in a good definition.

The amount of the release and solid-phase reagent layer 105 attached is not particularly limited, may be appropriately selected depending on the intended purpose, and is preferably 0.5 g/m² or greater but 50 g/m² or less. When the amount of the release and solid-phase reagent layer 105 attached is 0.5 g/m² or greater, the coating amount is appropriate and no deficiency is created in the release and solid-phase reagent layer 105 (resin layer 15). When the amount of the release and solid-phase reagent layer 105 attached is 50 g/m² or less, a drying time is appropriate and no unevenness is formed in the release and solid-phase reagent layer 105.

### —Formation of Reagent as Solid-Phase—

After the coating liquid is dried and the solid-phase reagent layer 103 or the release and solid-phase reagent layer 105 is formed, a solution containing the labeled antibody 16 or the capture antibody (17 or 18) is coated over the surface of the solid-phase reagent layer 103 or the release and solid-phase reagent layer 105, to form a uniform coating film. Subsequently, the coating film is dried. This makes it possible for the labeled antibody 16 or the capture antibody (17 or 18) to be formed as a solid phase over the surface of the solid-phase reagent layer 103 or the release and solid-phase reagent layer 105.

Further, in the formation of the capture antibody (17 or 18) as a solid phase, the surface over which the capture antibody has been formed as a solid phase is coated with the water-soluble resin in which the reagent is dispersed and then dried. As a result, the reagent deposited portion 20 can be formed over the surface.

### —Formation of Labeled Antibody as Solid Phase—

The Method for Forming the Labeled Antibody as a Solid Phase is not particularly limited and may be appropriately selected depending on the intended purpose. Examples of the method include a method of coating a coating liquid of the labeled antibody over the surface of the solid-phase reagent layer 103 or the release and solid-phase reagent layer 105 to form a water film and drying up the water film by, for example, natural drying, drying under reduced pressure, or freeze drying to form the water film as a solid phase.

It is preferable that the water film be coated to have a uniform thickness.

An amount of the labeled antibody coated is not particularly limited and may be appropriately selected depending on the intended purpose. For example, when using a gold colloid labeled antibody as the labeled antibody, it is preferable to coat a gold colloid labeled antibody having an OD (optical density) of from 1.0 through 30 in a coating amount of 20 microliters or greater but 600 microliters or less per unit area (cm²) of the resin layer. When the coating amount of the labeled antibody is 30 microliters or greater, the amount of the gold colloid labeled antibody is appropriate and a color developing intensity on a line is good. When the coating amount of the labeled antibody is 600 microliters or less, the amount of the gold colloid labeled antibody is appropriate and color development on a line is good.

The drying method is not particularly limited and may be appropriately selected depending on the intended purpose. Examples of the drying method include through-flow drying, vacuum drying, natural drying, and freeze drying. Among these drying methods, natural drying under a low humidity or drying under reduced pressure is preferable.

A humidity during drying is preferably 30% or lower on a relative humidity basis. When the relative humidity is 30% or lower, drying is appropriate and an antibody can be formed as a sufficiently solid phase.

It is preferable to perform drying at a drying temperature of from room temperature (20 degrees C.) through 50 degrees C. for a drying time of 30 minutes or longer but 24 hours or shorter. When the drying temperature is 20 degrees C. or higher, the drying time is appropriate and productivity is improved. When the drying temperature is 50 degrees C. or lower, the reagent can be prevented from being denatured by heat. When the drying time is 30 minutes or longer, drying can be performed appropriately. When the drying time is 24 hours or shorter, productivity is improved and discoloring can be prevented.

### —Formation of Capture Antibody as Solid Phase—

The method for forming the capture antibody as a solid phase is not particularly limited and may be appropriately selected depending on the intended purpose. Examples of the method include a method (dry-up method) of coating a coating liquid of the capture antibody over the surface of the solid-phase reagent layer 103 or the release and solid-phase reagent layer 105 to form a water film and drying up the water film by, for example, natural drying, drying under reduced pressure, or freeze drying to form the water film as a solid phase, and a method (adsorption drying method) of leaving the coating liquid standing still under a high humidity environment so as not for the coating liquid to dry, cleaning the surface of the solid-phase reagent layer 103 or the release and solid-phase reagent layer 105 with, for example, distilled water, and drying the coating liquid to a solid phase. In either case, it is preferable that the coating film be coated to have a uniform thickness.

The concentration in a buffer diluting the capture antibody is not particularly limited, and a buffer composition commonly used for diluting antibodies may be used. However, the concentration in the buffer is preferably 10 micrograms/mL or greater but 5,000 micrograms/mL or less and more preferably 100 micrograms/mL or greater but 1,000 micrograms/mL or less. When the coating concentration is 10 micrograms/mL or greater, the amount of the liquid coated per unit area (cm²) of the solid-phase reagent layer 103 or the release and solid-phase reagent layer 105 is appropriate. This suppresses the amount of components such as an inorganic salt contained in the buffer, and makes it possible to form the antibody as a sufficiently solid phase. When the coating concentration is 5,000 micrograms/mL or less, wettability and viscosity of the coating liquid are appropriate. This makes coating over the solid-phase reagent layer 103 or the release and solid-phase reagent layer 105 good, with an appropriate amount of the liquid coated, resulting in formation of a uniform water film.

The drying method used when forming the capture antibody as a solid phase by the dry-up method is not particularly limited and may be appropriately selected depending on the intended purpose. Examples of the drying method include through-flow drying, vacuum drying, natural drying, and freeze drying. Among these drying methods, natural drying under a low humidity or drying under reduced pressure is preferable.

The humidity during drying is preferably 30% or lower on a relative humidity basis. When the relative humidity is higher than 30%, there is a risk that drying is insufficient to make it impossible to form the antibody as a sufficiently solid phase.

It is preferable to perform drying at a drying temperature of from room temperature (20 degrees C.) through 50 degrees C. for a drying time of 30 minutes or longer but 24 hours or shorter. When the drying temperature is 20 degrees C. or higher, the drying time is appropriate and productivity is improved. When the drying temperature is 50 degrees C. or lower, the reagent can be prevented from being denatured by heat. When the drying time is 30 minutes or longer, drying can be performed appropriately. When the drying time is 24 hours or shorter, productivity is improved and discoloring can be prevented.

Preferable standing-still conditions when forming the capture antibody as a solid phase over the resin layer (15b or 15c) by the adsorption drying method include a temperature of 0 degree C. or higher but 40 degrees C. or lower and a relative humidity of 30% or higher. When the temperature is 0 degree C. or higher, it is possible to perform formation of the capture antibody as a solid phase appropriately. When the temperature is 40 degrees C. or lower, the capture antibody is not denatured. When the relative humidity is 30% or higher, water volatilization during standing still is low, and this prevents any undesirable component other than the antibody from being formed as a solid phase in a large amount.

The cleaning method after standing still is not particularly limited and may be appropriately selected depending on the intended purpose. Examples of the cleaning method include a method of pouring, for example, distilled water in an amount of 20 microliters or greater but 100 microliters or less per unit area (cm²) onto the surface for the solid phase formation using, for example, a shaker, and cleaning the surface at room temperature (25 degrees C.) by gentle shaking.

The drying method after cleaning is not particularly limited and may be appropriately selected depending on the intended purpose. Examples of the drying method include through-flow drying, vacuum drying, natural drying, and freeze drying. Among these drying methods, natural drying under a low humidity or drying under reduced pressure is preferable.

The humidity during drying is preferably 30% or lower on a relative humidity basis. When the relative humidity is 30% or lower, drying is appropriate and the antibody can be formed as a sufficiently solid phase. It is preferable to perform drying at a drying temperature of from room temperature (20 degrees C.) through 50 degrees C. for a drying time of 30 minutes or longer but 24 hours or shorter.

When the drying temperature is 20 degrees C. or higher, a drying time is appropriate and productivity is improved. When the drying temperature is 50 degrees C. or lower, the reagent can be prevented from being denatured by heat. When the drying time is 30 minutes or longer, drying can be performed appropriately. When the drying time is 24 hours or shorter, productivity is improved and discoloring of the resin can be prevented.

The amount of the capture antibody formed as a solid phase is preferably 500 ng/cm² or greater. When the amount of the capture antibody formed as a solid phase is 500 ng/cm² or greater, the amount of the capture antibody formed as a solid phase is appropriate and a sufficient color developing intensity can be obtained on a line.

Examples of the method for analyzing the amount of the antibody formed as a solid phase over the surface of the resin layer include X-ray photoelectron spectroscopy (XPS).

### <Reagent Deposited Portion>

The reagent deposited portion 20 is provided over the resin layers (resin layers 15b and 15c) that are provided with the solid-phase reagent over the surface of the resin layers.

The method for depositing the reagent deposited portion 20 over the resin layers (resin layers 15b and 15c) that are provided with the solid-phase reagent over the surface of the resin layers is not particularly limited and may be appropriately selected depending on the intended purpose. Examples of the method include a method of coating a coating liquid containing the water-soluble resin and the reagent such as the capture antibody over the resin layer 15b and the resin layer 15c and subsequently drying up the coating liquid by rapid drying.

### <Back Layer>

It is preferable that the transfer medium 100 for producing a testing device include a back layer 104 over a surface of the support 101 opposite to the surface of the support 101 provided with the release layer 102. During transfer, heat from, for example, a thermal head is directly applied to the opposite surface in a manner to match the shape of the resin layer. Therefore, it is preferable that the back layer 104 have resistance to a high heat and resistance to friction with, for example, the thermal head.

The back layer 104 contains a binder resin and further contains other components as needed.

The binder resin is not particularly limited and may be appropriately selected depending on the intended purpose. Examples of the binder resin include silicone-modified urethane resins, silicone-modified acrylic resins, silicone resins, silicone rubbers, fluororesins, polyimide resins, epoxy resins, phenol resins, melamine resins, and nitrocellulose. One of these binder resins may be used alone or two or more of these binder resins may be used in combination.

The other components are not particularly limited and may be appropriately selected depending on the intended purpose. Examples of the other components include inorganic particles of, for example, talc, silica, or organopolysiloxane, and a lubricant.

The method for forming the back layer 104 is not particularly limited and may be appropriately selected depending on the intended purpose. Examples of the method include a gravure coater, a wire bar coater, and a roll coater.

The average thickness of the back layer 104 is not particularly limited, may be appropriately selected depending on the intended purpose, and is preferably 0.01 micrometers or greater but 1.0 micrometer or less.

### <Undercoat Layer>

An undercoat layer may be provided between the support 101 and the release layer 102 and between the release layer 102 and the solid-phase reagent layer 103, or between the support 101 and the release and solid-phase reagent layer 105.

The undercoat layer contains a resin, and further contains other components as needed.

The resin is not particularly limited and may be appropriately selected depending on the intended purpose. Examples of the resin include the resins used in the solid-phase reagent layer 103, the release layer 102, and the release and solid-phase reagent layer 105.

### <Protective Film>

It is preferable to provide a protective film over the solid-phase reagent layer 103 or the release and solid-phase reagent layer 105 for protection from contamination and damage during storage.

The material of the protective film is not particularly limited and may be appropriately selected depending on the intended purpose so long as the material can be easily peeled from the solid-phase reagent layer 103 or the release and solid-phase reagent layer 105. Examples of the material of the protective film include silicone paper, polyolefin sheet such as of polypropylene, and polytetrafluoroethylene sheet.

The average thickness of the protective film is not particularly limited, may be appropriately selected depending on the intended purpose, and is preferably 5 micrometers or greater but 100 micrometers or less and more preferably 10 micrometers or greater but 30 micrometers or less.

Examples of the method for measuring the average thickness of each layer described above include a measuring method using the micrometer described above.

### <Testing Liquid>

When spreading a sample in the testing device, the testing liquid 30 has a function of smoothly chromatographing or spreading the sample throughout the flow path member to allow the sample to undergo a reaction with the solid-phase reagent over the resin layer efficiently.

The testing liquid 30 contains at least a sample, is preferably hydrophilic, and contains other components such as an inorganic salt, a surfactant, a sugar, and a protein as needed. Typically, a spreading liquid is prepared by adding, for example, a surfactant, a sugar, and a protein to the buffer solution described above, and a testing liquid is prepared by adding a sample to the spreading liquid. However, this is non-limiting.

### (Method for Producing Testing Device)

In a first embodiment, a method for producing a testing device of the present disclosure includes a step of bringing the solid-phase reagent layer of the transfer medium for producing a testing device of the first embodiment of the present disclosure into contact with a porous flow path member to transfer the solid-phase reagent layer onto the flow path member (this step may hereinafter be referred to as “solid-phase reagent layer transfer step”), and further includes other steps as needed.

By the transfer of the solid-phase reagent layer, the reagent reactive with the sample and the reagent deposited portion formed as solid phases over the surface of the solid-phase reagent layer are also transferred onto the flow path member.

In a second embodiment, a method for producing a testing device of the present disclosure includes a step of bringing the release and solid-phase reagent layer of the transfer medium for producing a testing device of the second embodiment of the present disclosure into contact with a porous flow path member to transfer the release and solid-phase reagent layer onto the flow path member (this step may hereinafter be referred to as “release and solid-phase reagent layer transfer step”), and further includes other steps as needed.

By the transfer of the release and solid-phase reagent layer, the reagent reactive with the sample and the reagent deposited portion formed as solid phases over the surface of the release and solid-phase reagent layer are also transferred onto the flow path member.

### <Solid-Phase Reagent Layer Transfer Step or Release and Solid-Phase Reagent Layer Transfer Step>

Examples of the method for thermally transferring the solid-phase reagent layer or the release and solid-phase reagent layer onto a flow path member include a method of bringing the solid-phase reagent layer or the release and solid-phase reagent layer of a transfer medium for a reagent into contact with a flow path member to transfer the solid-phase reagent layer or the release and solid-phase reagent layer onto the flow path member.

A printer used for the thermal transfer is not particularly limited and may be appropriately selected depending on the intended purpose. Examples of the printer include thermal printers equipped with, for example, a serial thermal head and a line-type thermal head.

Energy applied for the thermal transfer is not particularly limited, may be appropriately selected depending on the intended purpose, and is preferably 0.05 mJ/dot or higher but 0.5 mJ/dot or lower. When the applied energy is 0.05 mJ/dot or higher, it is possible to efficiently melt the solid-phase reagent layer 103 or the release and solid-phase reagent layer 105. When the applied energy is 0.5 mJ/dot or lower, it is possible to prevent the reagent from being thermally denatured. This prevents the support 101 from being dissolved or the thermal head from being contaminated.

### —Applications of Testing Device—

Applications of the testing device 10 are not particularly limited and may be appropriately selected depending on the intended purpose. Examples of the applications of the testing device 10 include biochemical sensors (sensing chips) for blood testing and DNA testing, and small-size analytical devices (chemical sensors) for, for example, quality control of foods and beverages.

Samples used in biochemical testings are not particularly limited and may be appropriately selected depending on the intended purpose. Examples of the samples include pathogens such as bacteria and viruses, and blood, saliva, lesional tissues, etc. separated from living organisms, and excretion such as enteruria. For performing a prenatal diagnosis, the sample may be a part of a fetus cell in an amniotic fluid or a part of a dividing egg cell in a test tube. These samples may be condensed to a sediment directly or by, for example, centrifugation as needed and then subjected to a pre-treatment for cell destruction by, for example, an enzymatic treatment, a thermal treatment, a surfactant treatment, an ultrasonic treatment, and any combinations of these treatments.

The testing device 10 of the present disclosure also has a function of chromatographing (separating or refining) the testing liquid because the flow path member 12 functions as a static bed. In this case, the flow path member 12 including the continuous cells of which internal wall has hydrophilicity functions as the static bed (or a support). Different components in the testing liquid 30 flow through the flow path at different speeds because of the difference in the interaction with the static bed during the process of permeating the flow path, i.e., the difference in whether the components are hydrophilic or hydrophobic.

A component having a higher hydrophilicity adsorbs to the porous portion functioning as the static bed more easily, and repeats adsorbing and desorbing more times, resulting in a lower speed of permeation through the flow path. In contrast, a component having a higher hydrophobicity permeates the flow path without adsorbing to the static bed, and hence moves through the flow path more quickly. By extracting the target component in the testing liquid 30 selectively based on the difference in the moving speed in the testing liquid and allowing the target component to undergo a reaction, it is possible to use the testing device 10 as a highly functional chemical or biochemical sensor.

### <Testing Method>

A testing method relating to the present disclosure is not particularly limited and may be appropriately selected depending on the intended purpose. The testing method includes a step of supplying a hydrophilic testing liquid to the flow path member of the testing device of the present disclosure and a step of bringing the labeled antibody (an example of a reagent) formed as a solid phase over the resin layer into contact with the testing liquid to release the labeled antibody from the resin layer. The testing method further includes other steps as needed.

A testing method using the testing device may include a step of supplying the testing liquid to the flow path member of the testing device and a step of making the capture antibody formed as a solid phase over the resin layer capture an antigen (an example of a part of a sample) when any antigen is contained in the testing liquid.

In a specific operation, the hydrophilic testing liquid 30 is dropped and supplied onto a dropping portion 12c (see FIG. 1) provided over the flow path member 12 of the testing device 10. Next, the supplied testing liquid 30 and the labeled antibody 16 formed as a solid phase over the resin layer 15a are brought into contact with each other, to release the labeled antibody 16 from the resin layer 15a. When any antigen 31 is contained in the testing liquid 30, the labeled antibody 16 released from the resin layer 15a reacts and binds with the antigen 31 (see FIG. 3). Next, the testing liquid 30 containing the labeled antibody 16 and the antigen 31 spreads along the flow path member 12 and arrives at the region at which the resin layer 15b is disposed. The capture antibody 17 formed as a solid phase over the surface of the resin layer 15b facing the flow path member 12 also binds with and captures the antigen 31 that is in the state of being bound with the labeled antibody 16. Further, the antibody dispersed in the water-soluble resin is gradually released toward the flow path member along with dissolution of the resin, to have an antigen-antibody reaction. The antigen-antibody reaction product quickly binds with the fiber constituting the flow path member 12, and does not flow out together with the testing liquid but remains in the membrane immediately below the capture antibody portion (line). The capture antibody 17 is formed as a solid phase over the resin layer 15b partially by the hydrophobic group 17g. Therefore, even when the capture antibody 17 contacts the testing liquid 30, the capture antibody 17 does not come to have affinity with the testing liquid 30 and is not easily released into the testing liquid 30. This facilitates immobilization of the labeled antibody 16 to about the resin layer 15b, resulting in a clear color development on the test line (see FIG. 4A to FIG. 4D).

Any labeled antibody 16 that passes by the resin layer 15b without being captured spreads along the flow path member 12 and arrives at the region at which the resin layer 15c is disposed. The capture antibody 18 containing a hydrophobic group and the reagent deposited portion 20 that is water-soluble are formed as solid phases over the surface of the resin layer 15c facing the flow path member 12. The labeled antibody 16 is captured by being bound with the capture antibody 18. Further, the antibody dispersed in the water-soluble resin is gradually released toward the flow path member along with dissolution of the resin, to have an antigen-antibody reaction. The antigen-antibody reaction product quickly binds with the fiber constituting the flow path member 12, and does not flow out together with the testing liquid 30 but remains in the membrane immediately below the capture antibody portion (line). The capture antibody 18 is formed as a solid phase over the resin layer 15c by the hydrophobic group. Therefore, even when the capture antibody 18 contacts the testing liquid 30, the capture antibody 18 does not come to have affinity with the testing liquid 30 and is not easily released into the testing liquid 30. This facilitates immobilization of the labeled antibody 16 to about the resin layer 15c, resulting in a clear color development on the control line (see FIG. 5A to FIG. 5D).

### (Testing Kit)

A testing kit of the present disclosure includes the testing device of the present disclosure, and at least one selected from the group consisting of a sample picking unit configured to pick a sample and a liquid for treating the sample, and further includes other members as needed.

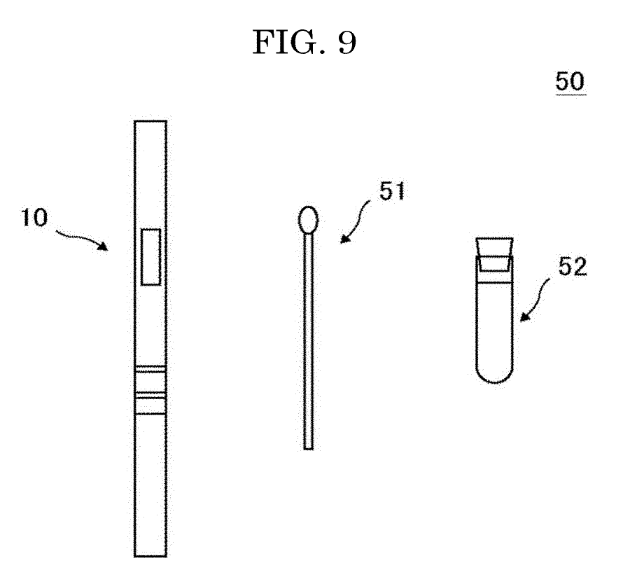

As illustrated in FIG. 9, the testing kit 50 includes the testing device 10 of the present disclosure and at least one of a tool configured to pick a sample (an example of the sample picking unit) and a liquid for treating the sample.

Examples of the tool configured to pick a sample include a sterilized cotton swab 51 for picking a sample from, for example, pharynx or nasal cavity.

Examples of the liquid for treating the sample include a diluting fluid 52 for diluting the sample and an extraction liquid for extracting the sample.

Examples of the other members include an instruction manual.

In the embodiment described above, a case where the reagent formed as a solid phase over the resin layer 15 is an antigen or an antibody is described. The present disclosure is not limited to this embodiment. The present disclosure can also be applied to, for example, a testing device using an indicator used in a chemical assay.

The indicator used in a chemical assay refers to a reagent for indicating a chemical property of a solution. The indicator is not particularly limited and may be appropriately selected depending on the intended purpose. Examples of the indicator include a pH indicator, various ionophores that discolor by reacting with various ions such as a lead ion, a copper ion, and a nitrite ion, and reagents that discolor by reacting with various agricultural chemicals.

In the embodiment described above, a case where the solid-phase reagent layer 103 or the release and solid-phase reagent layer 105 of the transfer medium 100 for producing a testing device is separated from the support 101 by heat during transfer is described. The present disclosure is not limited to this embodiment. For example, the solid-phase reagent layer 103 or the release and solid-phase reagent layer 105 may be separated from the support 101 by light. In this case, the release layer 102 or the release and solid-phase reagent layer 105 may contain a light absorber such as carbon black and may make the light absorber absorb light and generate heat, so that the release layer 102 or the release and solid-phase reagent layer 105 is fused to release the solid-phase reagent layer 103 or the release and solid-phase reagent layer 105. Alternatively, the release layer 102 or the release and solid-phase reagent layer 105 may contain a material that changes properties in response to light irradiation and may make the material absorb light, so that the release layer 102 is made fragile to release the solid-phase reagent layer 103 or the release and solid-phase reagent layer 105.

Examples of a transfer method other than the thermal transfer include a method of pasting a sheet formed of the solid-phase reagent layer 103 or the release and solid-phase reagent layer 105 over which the reagent is formed as a solid phase over the flow path member 12 by, for example, a tape.

In the embodiment described above, an example in which the flow path is formed throughout the flow path member 12 is described. The present disclosure is not limited to this embodiment. Examples of the method for forming a flow path in a partial region of the flow path member 12 include a method of forming a flow path wall defining an external edge of the flow path by filling the voids of the flow path member 12 with a hydrophobic material by a known method.

In the embodiment described above, an example in which the resin layers 15 are provided at a plurality of positions over the flow path member 12 is described. However, depending on the kind of the reagent, the resin layer 15 may be provided at one position over the flow path member 12. For example, a testing device capable of detecting a plurality of components at the same time can be obtained when the flow path member 12 that is provided with a resin layer 15a1 over which a reagent specifically bindable with a component A contained in the testing liquid 30 is formed as a solid phase and resin layers 15b1 and 15c1 over which reagents for capturing these reagent and component are formed as solid phases is further provided with a resin layer 15a2 over which a reagent specifically bindable with a component B contained in the testing liquid 30 is formed as a solid phase and resin layers 15b2 and 15c2 over which reagents for capturing these reagent and component are formed as solid phases.

In the embodiment described above, an example in which the testing liquid 30 is hydrophilic is described. However, the testing liquid is not limited to a hydrophilic liquid. For example, the testing liquid 30 may be a solvophilic liquid containing an organic solvent such as alcohols such as methyl alcohol, ethyl alcohol, 1-propyl alcohol, and 2-propyl alcohol, and ketones such as acetone and methyl ethyl ketone (MEK). In this case, the term “hydrophilic” in the embodiment described above is replaced by “hydrophobic”, and the term “hydrophobic” is replaced by “hydrophilic”.

### EXAMPLES

The present disclosure will be described by way of Examples. The present disclosure should not be construed as being limited to these Examples.

### Preparation Example 1

### —Preparation of Back Layer Coating Liquid—

A silicone-based rubber emulsion (available from Shin-Etsu Chemical Co., Ltd., KS779H, with a solid concentration of 30% by mass) (16.8 parts by mass), a chloroplatinic acid catalyst (0.2 parts by mass), and toluene (83 parts by mass) were mixed, to obtain a back layer coating liquid.

### Preparation Example 2

### —Preparation of Release and Solid-Phase Reagent Layer (for Immobilization) Coating Liquid—

A coating liquid (available from Ricoh Co., Ltd., B110AX stripping solution) containing a carnauba wax (90 parts by mass), an ethylene-vinyl acetate copolymer (1 part by mass), a styrene-butadiene copolymer (4 parts by mass), a butadiene rubber (4 parts by mass), an acrylonitrile-butadiene copolymer (1 part by mass), and a toluene/methyl ethyl ketone (at a ratio by volume of 7/3) solvent (600 parts by mass) was used as a release and solid-phase reagent layer (for immobilization) coating liquid of Preparation example 2.

### Preparation Example 3

### —Preparation of Solid-Phase Reagent Layer (for Release) Coating Liquid—

A polyvinyl butyral resin (available from Sekisui Chemical Co., Ltd., BL-1, with a butyralization degree of 64 mol %) (5 parts by mass) and ethanol (95 parts by mass) were mixed, to prepare a solid-phase reagent layer (for release) coating liquid of Preparation example 3.

### Preparation Example 4

### —Preparation of Test Line Reagent Coating Liquid—

As an antibody diluting fluid, a Dulbecco's phosphate buffered saline (free of Ca and Mg, D-PBS (−), available from Nacalai Tesque, Inc., 14249-95) was added to an anti-human IgG antibody (available from Sigma-Aldrich Co., LLC., 11886) to adjust the antibody concentration to 500 micrograms/mL, to prepare a test line reagent coating liquid of Preparation example 4.

### Preparation Example 5

### —Preparation of Test Line Reagent Depositing Liquid—

As a water-soluble resin, polyvinyl pyrrolidone (PVP, available from Tokyo Chemical Industry Co. Ltd., K90) was added to an anti-human IgG antibody prepared to 2 mg/mL with a Dulbecco's phosphate buffered saline in a manner that a solid content amount of the water-soluble resin became 0.5 mg per 1 mL of the anti-human IgG antibody, and sufficiently stirred, to prepare a test line reagent depositing liquid of Preparation example 5.

### Preparation Example 6

### —Preparation of Control Line Reagent Coating Liquid—

As a diluting fluid, a Dulbecco's phosphate buffered saline (free of Ca and Mg, D-PBS (−), available from Nacalai Tesque, Inc., 14249-95) was added to a human IgG (available from Sigma-Aldrich Co., LLC., I2511-10MG) to adjust the antibody concentration to 500 micrograms/mL, to prepare a control line reagent coating liquid of Preparation example 6.

### Preparation Example 7

### —Preparation of Control Line Reagent Depositing Liquid—

As a water-soluble resin, polyvinyl pyrrolidone (PVP, available from Tokyo Chemical Industry Co. Ltd., K90) was added to a human IgG prepared to 2 mg/mL with a Dulbecco's phosphate buffered saline in a manner that a solid content amount of the water-soluble resin became 0.5 mg per 1 mL of the IgG, and sufficiently stirred, to prepare a control line reagent depositing liquid of Preparation example 7.

### Preparation Example 8

### —Preparation of Test Line Reagent Depositing Liquid—

As a water-soluble resin, polyethylene oxide (available from Meisei Chemical Works, Ltd., ALKOX L-6) was added to an anti-human IgG antibody prepared to 2 mg/mL with a Dulbecco's phosphate buffered saline in a manner that a solid content amount of the water-soluble resin became 0.5 mg per 1 mL of the anti-human IgG antibody, and sufficiently stirred, to prepare a test line reagent depositing liquid of Preparation example 8.

### Preparation Example 9

### —Preparation of Control Line Reagent Depositing Liquid—

As a water-soluble resin, polyethylene oxide (available from Meisei Chemical Works, Ltd., ALKOX L-6) was added to a human IgG prepared to 2 mg/mL with a Dulbecco's phosphate buffered saline in a manner that a solid content amount of the water-soluble resin became 0.5 mg per 1 mL of the IgG, and sufficiently stirred, to prepare a control line reagent depositing liquid of Preparation example 9.

### Preparation Example 10

### —Preparation of Test Line Reagent Depositing Liquid—

As a water-soluble resin, an acrylic acid-based copolymer (available from Kuraray Co., Ltd., JULIMER AC 103AP) was added to an anti-human IgG antibody prepared to 2 mg/mL with a Dulbecco's phosphate buffered saline in a manner that a solid content amount of the water-soluble resin became 0.5 mg per 1 mL of the anti-human IgG antibody, and sufficiently stirred, to prepare a test line reagent depositing liquid of Preparation example 10.

### Preparation Example 11

### —Preparation of Control Line Reagent Depositing Liquid—

As a water-soluble resin, an acrylic acid-based copolymer (available from Kuraray Co., Ltd., JULIMER AC 103AP) was added to a human IgG prepared to 2 mg/mL with a Dulbecco's phosphate buffered saline in a manner that a solid content amount of the water-soluble resin became 0.5 mg per 1 mL of the IgG, and sufficiently stirred, to prepare a control line reagent depositing liquid of Preparation example 11.

### Preparation Example 12

### —Preparation of Release and Solid-Phase Reagent Layer (for Immobilization) Coating Liquid—

A coating liquid produced in the same manner as in Preparation example 2 except that unlike in Preparation example 2, the carnauba wax was changed to a polyethylene wax was used as a release and solid-phase reagent layer (for immobilization) coating liquid of Preparation example 12.

### Preparation Example 13

### —Preparation of Solid-Phase Reagent Layer (for Immobilization) Coating Liquid—

A polyvinyl butyral resin (available from Sekisui Chemical Co., Ltd., BL-10, with a butyralization degree of 72 mol %) (5 parts by mass) and ethanol (95 parts by mass) were mixed, to prepare a solid-phase reagent layer (for immobilization) coating liquid of Preparation example 13.

### Preparation Example 14

### —Preparation of Labeled Antibody Reagent Coating Liquid—

A gold colloid-labeled anti-human IgG antibody (available from Bioassay Works, LLC, Gold, with an average particle diameter of 40 nm, OD=15) was prepared as a labeled antibody reagent coating liquid.

### Preparation Example 15

### —Preparation of Test Line Reagent Coating Liquid—

As an antibody diluting fluid, a Dulbecco's phosphate buffered saline (free of Ca and Mg, D-PBS (−), available from Nacalai Tesque, Inc., 14249-95) was added to an anti-hCG monoclonal antibody (available from Medix Biochemica Inc., ANTI-ALPHA SUBUNIT 6601 SPR-5) to adjust the antibody concentration to 500 micrograms/mL, to prepare a test line reagent coating liquid of Preparation example 15.

### Preparation Example 16

### —Preparation of Test Line Reagent Depositing Liquid—

As a water-soluble resin, polyvinyl pyrrolidone (PVP, available from Tokyo Chemical Industry Co. Ltd., K90) was added to an anti-hCG monoclonal antibody prepared to 2 mg/mL with a Dulbecco's phosphate buffered saline in a manner that a solid content amount of the water-soluble resin became 0.5 mg per 1 mL of the anti-hCG monoclonal antibody, and sufficiently stirred, to prepare a test line reagent depositing liquid of Preparation example 16.

### Preparation Example 17

### —Preparation of Control Line Reagent Coating Liquid—

As an antibody diluting fluid, a Dulbecco's phosphate buffered saline (free of Ca and Mg, D-PBS (−), available from Nacalai Tesque, Inc., 14249-95) was added to an anti-mouse IgG antibody (available from Wako Pure Chemical Industries, Ltd., 566-70621) to be adjusted to 500 micrograms/mL, to prepare a control line reagent coating liquid of Preparation example 17.

### Preparation Example 18

### —Preparation of Control Line Reagent Depositing Liquid—

As a water-soluble resin, polyvinyl pyrrolidone (PVP, available from Tokyo Chemical Industry Co. Ltd., K90) was added to an anti-mouse IgG antibody prepared to 2 mg/mL with a Dulbecco's phosphate buffered saline in a manner that a solid content amount of the water-soluble resin became 0.5 mg per 1 mL of the anti-mouse IgG antibody, and sufficiently stirred, to prepare a control line reagent depositing liquid of Preparation example 18.

### Preparation Example 19

### —Preparation of Labeled Antibody Reagent Coating Liquid—

A KH₂PO₄ buffer (with pH of 7.0) prepared to 50 mM (1 mL), and subsequently an anti-hCG monoclonal antibody (available from Medix Biochemica Inc., anti-hCG 5008 SP-5) prepared to 50 micrograms/mL (1 mL) were added to a gold colloid solution (available from BBI Solutions Inc., EMGC50) (9 mL) and stirred. The resultant was left to stand still for 10 minutes. To the resultant, a 1% by mass polyethylene glycol aqueous solution (available from Wako Pure Chemical Industries, Ltd., 168-11285) (550 microliters) was added and stirred, and then a 10% by mass BSA aqueous solution (available from Sigma-Aldrich Co., LLC., A-7906) (1.1 mL) was added and stirred.

Subsequently, this solution was centrifuged for 30 minutes. The supernatant was removed from this solution except for about 1 mL of the supernatant. The resultant solution was subjected to gold colloid re-dispersion with an ultrasonic cleaning machine. The centrifugation was performed with a centrifuge (available from Hitachi Koki Co., Ltd., HIMAC CF16RN) at a centrifugal acceleration of 8,000×g at 4 degrees C. Subsequently, the solution was dispersed in a gold colloid preservative solution (20 mM Tris-HCl buffer (with pH of 8.2), 0.05% by mass polyethylene glycol, 150 mM NaCl, a 1% by mass BSA aqueous solution, and a 0.1% by mass NaN₃ aqueous solution) (20 mL) and again centrifuged under the same conditions as described above. Subsequently, the supernatant was removed except for about 1 mL. The resultant solution was subjected to gold colloid re-dispersion with an ultrasonic cleaning machine. These operations were repeated to adjust the optical density (OD) of the gold colloid preservative solution to 15, to obtain a labeled antibody reagent coating liquid of Preparation example 19.

### Example 1

### <Production of Transfer Medium for Test Line>

### —Formation of Back Layer—

The back layer coating liquid of Preparation example 1 was coated over one surface of a support, which was a polyethylene terephthalate (PET) film having an average thickness of 4.5 micrometers (available from Toray Industries, Inc., LUMIRROR F57), and dried at 80 degrees C. for 10 seconds, to form a back layer having an average thickness of 0.02 micrometers.

### —Formation of Release and Solid-Phase Reagent Layer (for Immobilization)—

Next, the release and solid-phase reagent layer (for immobilization) coating liquid of Preparation example 2 was coated over a surface of the PET film opposite to the surface over which the back layer was formed, and dried at 40 degrees C. for 10 seconds, to form a release and solid-phase reagent layer having an average thickness of 20 micrometers.

### —Formation of Reagent as Solid Phase—

Subsequently, the test line reagent coating liquid of Preparation example 4 was coated over the release and solid-phase reagent layer (for immobilization) in an amount of 12 microliters per unit area (cm²) to form a water film. Subsequently, the transfer medium was set in a container maintained at a relative humidity of 80% so as not for the water film to dry, and left to stand still at 25 degrees C. for 10 minutes. After standing still, the surface of the release and solid-phase reagent layer of the transfer medium was cleaned under the conditions described below.

### —Cleaning—

The transfer medium peeled from the water film was pasted over a shaker (SHAKE-XR mounted with WR-3636, both available from Taitec Corporation), in a manner that the surface over which the reagent was to be formed as a solid phase faced outside.

Subsequently, distilled water was poured onto the surface over which the reagent was to be formed as a solid phase in an amount of 100 microliters per unit area (cm²) of the surface, and then the transfer medium was gently shaken at 25 degrees C. at a shaking speed of 20 r/min for 1 minute. After shaking was completed, the supernatant liquid after cleaning was removed. This operation was repeated a total of 5 times. Finally, the supernatant liquid after cleaning was sufficiently drained off from the surface of the transfer medium, for cleaning to be completed (number of times of cleaning: 5 times). After cleaning, the transfer medium was dried in a dessicator of a temperature of 25 degrees C. and a relative humidity of 20% for 15 minutes, to form the reagent as a solid phase over the release and solid-phase reagent layer (for immobilization).

### —Formation of Reagent Deposited Portion—

Subsequently, the test line reagent depositing liquid of Preparation example 5 was coated over the surface of the release and solid-phase reagent layer over which the reagent was formed as a solid phase in an amount of 200 microliters per unit area (cm²) of the surface, and subjected to vacuum drying at room temperature (25 degrees C.) for 2 hours, to form a reagent deposited portion having an average thickness of 5 micrometers. In the way described above, a transfer medium for a test line of Example 1 was obtained.

### <Production of Transfer Medium for Control Line>

A transfer medium for a control line of Example 1 was obtained in the same manner as in “Production of transfer medium for test line” described above, except that unlike in the process from “Formation of reagent as solid phase” to “Formation of reagent deposited portion” in “Production of transfer medium for test line”, the test line reagent coating liquid of Preparation example 4 was changed to the control line reagent coating liquid of Preparation example 6, and the test line reagent depositing liquid of Preparation example 5 was changed to the control line reagent depositing liquid of Preparation example 7.

### <Production of Transfer Medium for Labeled Antibody>

In the same manner as in “Production of transfer medium for test line”, a back layer and a release and solid-phase reagent layer (for immobilization) were formed over one surface of a support, which was a polyethylene terephthalate (PET) film having an average thickness of 4.5 micrometers (available from Toray Industries, Inc., LUMIRROR F57). Subsequently, the solid-phase reagent layer (for release) coating liquid of Preparation example 3 was coated over the release and solid-phase reagent layer (for immobilization) and dried at 40 degrees C. for 10 minutes, to form a solid-phase reagent layer (for release) having an average thickness of 5 micrometers.

Subsequently, the labeled antibody reagent coating liquid of Preparation example 14 was coated over the solid-phase reagent layer (for release) in an amount of 30 microliters/cm² and dried in a vacuum dryer at 25 degrees C. for 5 hours, to form the reagent as a solid phase over the solid-phase reagent layer (for release). In the way described above, the transfer medium for a labeled antibody of Example 1 was obtained.

### <Production of Testing Device>

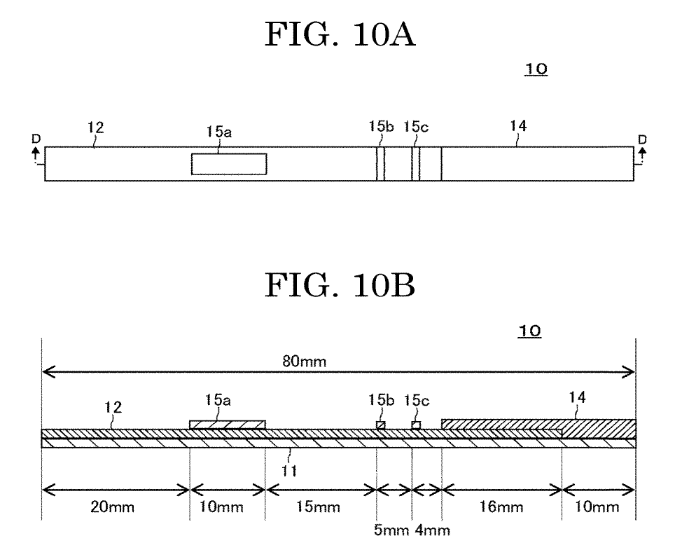

In the manner described below, a testing device 10 illustrated in FIG. 10A and FIG. 10B was produced. FIG. 10A is a top view of the testing device of Example 1. FIG. 10B is a cross-sectional view of the testing device of FIG. 10A taken along a line D-D.

### —Production of Paper Substrate—

As a thermoplastic resin, a polyester-based hot-melt adhesive (available from Toagosei Co., Ltd., ARONMELT PES375S40) was heated to 190 degrees C., and then with a roll coater, coated over a base material 11, which was a PET film (available from Toray Industries, Inc., LUMIRROR S10, with an average thickness of 50 micrometers) cut into a size of 40 mm in width and 80 mm in length, to have a thickness of 50 micrometers over the PET film, to form an adhesive layer.

The PET film over which the adhesive layer was formed was left to stand still for 2 hours or longer. Subsequently, a nitrocellulose membrane (available from Merck Millipore Corporation, HF180, with a voidage of 70%) cut into a size of 40 mm in width and 70 mm in length was overlapped over the adhesive layer-coated surface in a manner that one end of the adhesive layer-coated surface in the longer direction and one end of each member in the longer direction (this end is referred to as upstream end, and the opposite end is referred to as downstream end) would coincide with each other, and a load of 1 kgf/cm² was applied to the overlapped members at a temperature of 150 degrees C. for 10 seconds. Finally, the obtained product was cut along the longer direction into a size of 4 mm in width and 80 mm in length, to obtain a paper substrate, which was a flow path member 12.

The voidage of the paper substrate, which was the flow path member 12, was calculated according to a calculation formula 1 below based on the basis weight (g/m²) of the paper substrate, the thickness (micrometer) of the paper substrate, and specific gravity of the component of the paper substrate. As a result, the voidage of the paper substrate was 70%. A paper substrate having a voidage of 40% or higher but 90% or lower can be said to be a porous paper substrate.

Voidage (%)={1−[basis weight (g/m²)/thickness (micrometer)/specific gravity of the component]}×100 [Calculation formula 1]

### —Transfer of Labeled Antibody—

The paper substrate, which was the flow path member 12, and the surface of the transfer medium for a labeled antibody having the reagent formed as a solid phase was faced and overlapped with each other. Subsequently, with a thermal transfer printer, the transfer medium for a labeled antibody was transferred in a pattern of 3 mm in width and 10 mm in length (resin layer 15a) onto the paper substrate at a position away from the upstream end by 20 mm, as illustrated in FIG. 10A and FIG. 10B.

The thermal transfer printer was equipped with a thermal head having a dot density of 300 dpi (available from TDK Corporation), and constructed as an evaluation system having a printing speed of 8.7 mm/sec and an applied energy of 0.35 mJ/dot.

### —Transfer of Test Line and Control Line—

As illustrated in FIG. 10A and FIG. 10B, the reagent deposited portion 20 of the transfer medium for a test line was overlapped at a position that was away by 15 mm from the position to which the transfer medium for a labeled antibody was transferred, and transferred to the position in a line shape of 4 mm in height and 1 mm in length (resin layer (test line) 15b). Then, the reagent deposited portion 20 of the transfer medium for a control line was overlapped at a position that was away by 5 mm from the position to which the transfer medium for a test line was transferred, and transferred to the position in a line shape of 4 mm in height and 1 mm in length (resin layer (control line) 15c). The resin layers (lines) were formed under the same printing conditions as used in “Transfer of labeled antibody” described above.

### —Production of Absorbing Member—

An absorbing member 14 (available from Merck Millipore Corporation, SUREWICK C248) was provided as illustrated in FIG. 10A and FIG. 10B, to obtain an immunochromatoassay (testing device 10) of Example 1.

### <Evaluation of Line>

### —Preparation of Testing Liquid—

As a carrier liquid, a D-PBS (−) solution of 0.1% by mass TWEEN 20 (available from Sigma-Aldrich Co., LLC., P9416-50ML) was prepared.

Subsequently, the carrier liquid was added to human IgG to adjust the antibody concentration to 500 ng/mL, to obtain a testing liquid.

### —Reaction 1 (Signal)—

The testing liquid (100 microliters) was dropped onto the upstream end of the immunochromatoassay illustrated in FIG. 10A and FIG. 10B. Fifteen minutes later, the test line was observed.

Next, the immunochromatoassay in which the reaction was completed was stored in a housing case for measurement, and the line density was measured with a chromatoreader (available from Otsuka Electronics Co., Ltd., DIASCAN 10), to obtain the color developing density on the line as a reading. The reading was evaluated according to the criteria described below. The result is presented in Table 2. A greater reading is more preferable, because the color development on the line was denser.

### [Evaluation Criteria]

A: The reading was 230 or greater.

B: The reading was 200 or greater but less than 230

C: The reading was 100 or greater but less than 200.

D: The reading was less than 100 or unmeasurable.

### —Reaction 2 (Non-Specific Adsorption)—

The testing liquid (100 microliters) was dropped onto the upstream end of the immunochromatoassay illustrated in FIG. 10A and FIG. 10B. Fifteen minutes later, the test line was observed.

Next, the immunochromatoassay in which spreading was completed was stored in a housing case for measurement, and the line density was measured with a chromatoreader (available from Otsuka Electronics Co., Ltd., DIASCAN 10), to confirm presence or absence of color development due to non-specific adsorption. The result is presented in Table 2.

### [Evaluation Criteria]

A: No color was developed (no line was detected).

B: Color development was recognized (a reading was obtained).

### —Evaluation of Color Development on Surface of Flow Path Member—

The line portion (resin layer) of the testing device 10 in which the reaction was completed was peeled to visually observe and evaluate whether a color was developed on the flow path member (membrane) according to the criteria described below. The result is presented in Table 2.

### [Evaluation Criteria]

A: A color was developed.

B: Almost no color development was recognized.

### —Evaluation of Color Development on Surface of Resin Layer—

The line portion (resin layer) of the testing device 10 in which the reaction was completed was peeled to visually observe and evaluate whether a color was developed on the solid-phase reagent surface of the resin layer according to the criteria described below. The result is presented in Table 2.

### [Evaluation Criteria]

A: A color was developed.

B: Almost no color was developed.

### Example 2

### <Production of Transfer Medium for Test Line>

A transfer medium for a test line of Example 2 was obtained in the same manner as in Example 1, except that unlike in “Formation of reagent deposited portion” in “Production of transfer medium for test line” in Example 1, the test line reagent depositing liquid of Preparation example 8 was coated instead of the test line reagent depositing liquid of Preparation example 5.

### <Production of Transfer Medium for Control Line>

A transfer medium for a control line of Example 2 was obtained in the same manner as in Example 1, except that unlike in “Formation of reagent deposited portion” in “Production of transfer medium for control line” in Example 1, the control line reagent depositing liquid of Preparation example 9 was coated instead of the control line reagent depositing liquid of Preparation example 7.

An immunochromatoassay (testing device 10) of Example 2 was produced in the same manner as in Example 1 except the steps described above and subjected to the same evaluations as in Example 1. The results are presented in Table 2.

### Example 3

### <Production of Transfer Medium for Test Line>

A transfer medium for a test line of Example 3 was obtained in the same manner as in Example 1, except that unlike in “Formation of reagent deposited portion” in “Production of transfer medium for test line” in Example 1, the reagent depositing liquid of Preparation example 10 was coated instead of the reagent depositing liquid of Preparation example 5.

### <Production of Transfer Medium for Control Line>

A transfer medium for a control line of Example 3 was obtained in the same manner as in Example 1, except that unlike in “Formation of reagent deposited portion” in “Production of transfer medium for control line” in Example 1, the control line reagent depositing liquid of Preparation example 11 was coated instead of the control line reagent depositing liquid of Preparation example 7.

An immunochromatoassay (testing device 10) of Example 3 was produced in the same manner as in Example 1 except the steps described above and subjected to the same evaluations as in Example 1. The results are presented in Table 2.

### Example 4

### <Production of Transfer Medium for Test Line>

A transfer medium for a test line of Example 4 was obtained in the same manner as in “Production of transfer medium for test line” in Example 1, except that unlike in “Production of transfer medium for test line” in Example 1, the test line reagent coating liquid of Preparation example 4 was changed to the test line reagent coating liquid of Preparation example 15, and the test line reagent depositing liquid of Preparation example 5 was changed to the test line reagent depositing liquid Preparation example 16.

### <Production of Transfer Medium for Control Line>

Next, a transfer medium for a control line of Example 4 was obtained in the same manner as in “Production of transfer medium for control line” in Example 1, except that unlike in “Production of transfer medium for control line” in Example 1, the control line reagent coating liquid of Preparation example 6 was changed to the control line reagent coating liquid of Preparation example 17, and the control line reagent depositing liquid of Preparation example 7 was changed to the control line reagent depositing liquid Preparation example 18.

### <Production of Transfer Medium for Labeled Antibody>

A transfer medium for a labeled antibody of Example 4 was obtained in the same manner as in “Production of transfer medium for labeled antibody” in Example 1, except that unlike in “Production of transfer medium for labeled antibody” in Example 1, the labeled antibody coating liquid of Preparation example 14 was changed to the labeled antibody coating liquid of Preparation example 19.

An immunochromatoassay (testing device 10) of Example 4 was produced in the same manner as in Example 1 except the steps described above.

<Evaluation of line>

### —Preparation of Testing Liquid—

As a carrier liquid, a D-PBS (−) solution of 0.1% by mass TWEEN 20 (available from Sigma-Aldrich Co., LLC., P9416-50ML) was prepared.

Subsequently, the carrier liquid was added to hCG (available from R&D Systems Inc., RECOMBINANT HCG, 7727-CG-010) to adjust the antibody concentration to 500 mIU/mL, to obtain a testing liquid.

### —Reaction 1 (Signal)—

The testing liquid (100 microliters) was dropped onto the upstream end of the immunochromatoassay illustrated in FIG. 10A and FIG. 10B. Fifteen minutes later, the test line was observed.

Next, the line density was evaluated in the same manner as in Example 1. The result is presented in Table 2.

### —Reaction 2 (Non-Specific Adsorption)—

The testing liquid (100 microliters) was dropped onto the upstream end of the immunochromatoassay illustrated in FIG. 10A and FIG. 10B. Fifteen minutes later, the test line was observed.

Next, the line density was measured in the same manner as in Example 1 to confirm presence or absence of color development due to non-specific adsorption. The result is presented in Table 2.

### —Evaluation of Color Development on Surface of Flow Path Member—

Evaluation of color development on the surface of the flow path member was performed in the same manner as in Example 1. The result is presented in Table 2.

### —Evaluation of Color Development on Surface of Resin Layer—

Evaluation of color development on the surface of the resin layer was performed in the same manner as in Example 1. The result is presented in Table 2.

### Example 5

### <Production of Transfer Medium for Test Line>

A transfer medium for a test line of Example 5 was obtained in the same manner as in “Production of transfer medium for test line” in Example 1, except that unlike in the step of “Formation of release and solid-phase reagent layer (for immobilization)” in “Production of transfer medium for test line” in Example 1, the release and solid-phase reagent layer (for immobilization) coating liquid of Preparation example 12 was used instead of the release and solid-phase reagent layer (for immobilization) coating liquid of Preparation example 2.

### <Production of Transfer Medium for Control Line>

Next, a transfer medium for a control line of Example 5 was obtained in the same manner as in “Production of transfer medium for control line” in Example 1, except that unlike in the step of “Formation of release and solid-phase reagent layer (for immobilization)” in “Production of transfer medium for control line” in Example 1, the release and solid-phase reagent layer (for immobilization) coating liquid of Preparation example 12 was used instead of the release and solid-phase reagent layer (for immobilization) coating liquid of Preparation example 2. An immunochromatoassay (testing device 10) of Example 5 was produced in the same manner as in Example 1 except the steps described above and subjected to the same evaluations as in Example 1. The results are presented in Table 2.

### Example 6

### <Production of Transfer Medium for Test Line>

A transfer medium for a test line of Example 6 was obtained in the same manner as in Example 1, except that the steps until before “Formation of reagent as solid phase” in “Production of transfer medium for test line” in Example 1 were changed to the steps described below.

### —Formation of Back Layer—

The back layer coating liquid of Preparation example 1 was coated over one surface of a support, which was a polyethylene terephthalate (PET) film having an average thickness of 4.5 micrometers (available from Toray Industries, Inc., LUMIRROR F57), and dried at 80 degrees C. for 10 seconds, to form a back layer having an average thickness of 0.02 micrometers.

### —Formation of Release and Solid-Phase Reagent Layer (Release Layer)—

Next, the release and solid-phase reagent layer (for immobilization) coating liquid of Preparation example 2 was coated over a surface of the PET film opposite to the surface over which the back layer was formed, and dried at 40 degrees C. for 10 minutes, to form a release and solid-phase reagent layer (release layer) having an average thickness of 20 micrometers.

### —Formation of Solid-Phase Reagent Layer (for Immobilization)—

Next, the solid-phase reagent layer (for immobilization) coating liquid of Preparation example 13 was coated over the surface over which the release and solid-phase reagent layer (release layer) was formed, and dried at 40 degrees C. for 10 minutes, to form a solid-phase reagent layer having an average thickness of 5 micrometers.

A transfer medium for a test line of Example 6 was obtained in the same manner as in “Production of transfer medium for test line” in Example 1 except the above.

### <Production of Transfer Medium for Control Line>

A transfer medium for a control line of Example 6 was obtained in the same manner as in Example 1, except that the steps until before “Formation of reagent as solid phase” in “Production of transfer medium for control line” in Example 1 were changed to the steps described above.

An immunochromatoassay (testing device 10) of Example 6 was produced in the same manner as in Example 1 except the steps described above and subjected to the same evaluations as in Example 1. The results are presented in Table 2.

### Comparative Example 1

### <Production of Transfer Medium for Test Line>

### —Formation of Back Layer—

The back layer coating liquid of Preparation example 1 was coated over one surface of a support, which was a polyethylene terephthalate (PET) film having an average thickness of 4.5 micrometers (available from Toray Industries, Inc., LUMIRROR F57), and dried at 80 degrees C. for 10 seconds, to form a back layer having an average thickness of 0.02 micrometers.

### —Formation of Release and Solid-Phase Reagent Layer (for Immobilization)—

Next, the release and solid-phase reagent layer (for immobilization) coating liquid of Preparation example 2 was coated over a surface of the PET film opposite to the surface over which the back layer was formed, and dried at 40 degrees C. for 10 minutes, to form a release and solid-phase reagent layer having an average thickness of 20 micrometers.

### —Production of Transfer Medium for Test Line—

Subsequently, the test line reagent coating liquid of Preparation example 4 was coated over the release and solid-phase reagent layer (for immobilization) in an amount of 12 microliters per unit area (cm²) to form a water film. Subsequently, the transfer medium was set in a container maintained at a relative humidity of 80% so as not for the water film to dry, and left to stand still at 25 degrees C. for 10 minutes. After standing still, the surface of the release and solid-phase reagent layer of the transfer medium was cleaned under the conditions described below.

### —Cleaning—

The transfer medium peeled from the water film was pasted over a shaker (SHAKE-XR mounted with WR-3636, both available from Taitec Corporation), in a manner that the surface over which the reagent was to be formed as a solid phase faced outside.

Subsequently, distilled water was poured onto the surface over which the reagent was to be formed as a solid phase in an amount of 100 microliters per unit area (cm²) of the surface, and then the transfer medium was gently shaken at 25 degrees C. at a shaking speed of 20 r/min for 1 minute. After shaking was completed, the supernatant liquid after cleaning was removed. This operation was repeated a total of 5 times. Finally, the supernatant liquid after cleaning was sufficiently drained off from the surface of the transfer medium, for cleaning to be completed (number of times of cleaning: 5 times). After cleaning, the transfer medium was dried in a dessicator of a temperature of 25 degrees C. and a relative humidity of 20% for 15 minutes, to form the reagent as a solid phase over the release and solid-phase reagent layer (for immobilization). In the way described above, a transfer medium for a test line of Comparative Example 1 was obtained.

### <Production of Transfer Medium for Control Line>

A transfer medium for a control line of Comparative Example 1 was obtained in the same manner as in “Production of transfer medium for test line” described above, except that unlike in “Production of transfer medium for test line”, the test line reagent coating liquid of Preparation example 4 was changed to the control line reagent coating liquid of Preparation example 6.

An immunochromatoassay (testing device 10) of Comparative Example 1 was produced in the same manner as in Example 1 except the steps described above and subjected to the same evaluations as in Example 1. The results are presented in Table 2.

### Comparative Example 2

### <Production of Transfer Medium for Test Line>

### —Formation of Back Layer—

The back layer coating liquid of Preparation example 1 was coated over one surface of a support, which was a polyethylene terephthalate (PET) film having an average thickness of 4.5 micrometers (available from Toray Industries, Inc., LUMIRROR F57), and dried at 80 degrees C. for 10 seconds, to form a back layer having an average thickness of 0.02 micrometers.

### —Formation of Release and Solid-Phase Reagent Layer (for Immobilization)—

Next, the release and solid-phase reagent layer (for immobilization) coating liquid of Preparation example 2 was coated over a surface of the PET film opposite to the surface over which the back layer was formed, and dried at 40 degrees C. for 10 minutes, to form a release and solid-phase reagent layer having an average thickness of 20 micrometers.

### —Formation of Reagent Deposited Portion—

Subsequently, the reagent depositing liquid of Preparation example 5 was coated over the release and solid-phase reagent layer (for immobilization) in an amount of 200 microliters per unit area (cm²), and subjected to vacuum drying at room temperature (25 degrees C.) for 2 hours, to form a reagent deposited portion having an average thickness of 5 micrometers. In the way described above, a transfer medium for a test line of Comparative Example 2 was obtained.

An immunochromatoassay (testing device 10) of Comparative Example 2 was produced in the same manner as in Example 1 except the steps described above and subjected to the same evaluations as in Example 1. The results are presented in Table 2.

The details of the testing devices of Examples 1 to 6 and Comparative Examples 1 and 2 are presented in Table 1-1 and Table 1-2 collectively.

TABLE 1-1

| | Test line (second resin layer) | | | | Control line (second resin layer) | | | |
|---|---|---|---|---|---|---|---|---|
| | | | Reagent | | Release | | | |
| | Release and | | deposited portion | | and | Control | Reagent deposited portion | |
| | solid-phase | Test line | | Average | solid-phase | line | | Average |
| | reagent | coating | | thickness | reagent | coating | | thickness |
| | layer | liquid | Kind | (micrometer) | layer | liquid | Kind | (micrometer) |
| Ex. 1 | Prep. ex. 2 | Prep. ex. 4 | Prep. ex. 5 | 5 | Prep. ex. 2 | Prep. ex. 6 | Prep. ex. 7 | 5 |
| | (coating | | (PVP) | | (coating | | (PVP) | |
| | liquid A) | | | | liquid A) | | | |
| Ex. 2 | Prep. ex. 2 | Prep. ex. 4 | Prep. ex. 8 | 5 | Prep. ex. 2 | Prep. ex. 6 | Prep. ex. 9 | 5 |
| | (coating | | (PEG) | | (coating | | (PEG) | |
| | liquid A) | | | | liquid A) | | | |
| Ex. 3 | Prep. ex. 2 | Prep. ex. 4 | Prep. ex. | 5 | Prep. ex. 2 | Prep. ex. 6 | Prep. ex. 11 | 5 |
| | (coating | | 10 (AC) | | (coating | | (AC) | |
| | liquid A) | | | | liquid A) | | | |
| Ex. 4 | Prep. ex. 2 | Prep. ex. | Prep. ex. | 5 | Prep. ex. 2 | Prep. ex. | Prep. ex. 18 | 5 |
| | (coating | 15 | 16 (PVP) | | (coating | 17 | (PVP) | |
| | liquid A) | | | | liquid A) | | | |
| Ex. 5 | Prep. ex. 12 | Prep. ex. 4 | Prep. ex. 5 | 5 | Prep. ex. | Prep. ex. 6 | Prep. ex. 7 | 5 |
| | (coating | | (PVP) | | 12 (coating | | (PVP) | |
| | liquid B) | | | | liquid B) | | | |
| Ex. 6 | Prep. ex. | Prep. ex. 4 | Prep. ex. 5 | 5 | Prep. ex. | Prep. ex. 6 | Prep. ex. 7 | 5 |
| | 2 + Prep. ex. | | (PVP) | | 2 + Prep. ex. | | (PVP) | |
| | 13 | | | | 13 | | | |
| | (coating | | | | (coating | | | |
| | liquid | | | | liquid | | | |
| | A + coating | | | | A + coating | | | |
| | liquid C) | | | | liquid C) | | | |
| Comp. | Prep. ex. 2 | Prep. ex. 4 | — | — | Prep. ex. 2 | Prep. ex. 6 | — | — |
| Ex. 1 | (coating | | | | (coating | | | |
| | liquid A) | | | | liquid A) | | | |
| Comp. | Prep. ex. 2 | — | Prep. ex. 5 | 5 | Prep. ex. 2 | — | Prep. ex. 7 | 5 |
| Ex. 2 | (coating | | (PVP) | | (coating | | (PVP) | |
| | liquid A) | | | | liquid A) | | | |

*PVP: polyvinyl pyrrolidone: a water-soluble resin
*PEG: polyethylene oxide; a water-soluble resin
*AC: acrylic acid-based copolymer; a water-soluble resin
*Coating liquid A (Preparation example 2): a coating liquid containing a water-insoluble resin, a coating liquid (available from Ricoh Co., Ltd., B110AX stripping solution) containing a carnauba wax (90 parts by mass), an ethylene-vinyl acetate copolymer (1 part by mass), a styrene-butadiene copolymer (4 parts by mass), a butadiene rubber (4 parts by mass), an acrylonitrile-butadiene copolymer (1 part by mass), and a toluene/methyl ethyl ketone (at a ratio by volume of 7/3) solvent (600 parts by mass)
*Coating liquid B (Preparation example 12): a coating liquid containing a water-insoluble resin, a coating liquid containing a polyethylene wax (90 parts by mass), an ethylene-vinyl acetate copolymer (1 part by mass), a styrene-butadiene copolymer (4 parts by mass), a butadiene rubber (4 parts by mass), an acrylonitrile-butadiene copolymer (1 part by mass), and a toluene/methyl ethyl ketone (at a ratio by volume of 7/3) solvent (600 parts by mass)
*Coating liquid C (Preparation example 13): a coating liquid containing a water-insoluble resin, a coating liquid containing a polyvinyl butyral resin (available from Sekisui Chemical Co., Ltd., BL-10, with a butyralization degree of 72 mol %) (5 parts by mass) and ethanol (95 parts by mass)
*Coating liquid A + Coating liquid C (Preparation example 2 + Prepration example 13): lamination of a layer of the coating liquid A and a layer of the coating liquid C

TABLE 1-2

| | First resin layer | |
|---|---|---|
| | Solid-phase reagent layer | Labeled antibody coating liquid |
| Ex. 1 | Preparation example 3 | Preparation example 14 |
| | (coating liquid D) | |
| Ex. 2 | Preparation example 3 | Preparation example 14 |
| | (coating liquid D) | |
| Ex. 3 | Preparation example 3 | Preparation example 14 |
| | (coating liquid D) | |
| Ex. 4 | Preparation example 3 | Preparation example 19 |
| | (coating liquid D) | |
| Ex. 5 | Preparation example 3 | Preparation example 14 |
| | (coating liquid D) | |
| Ex. 6 | Preparation example 3 | Preparation example 14 |
| | (coating liquid D) | |
| Comp. | Preparation example 3 | Preparation example 14 |
| Ex. 1 | (coating liquid D) | |
| Comp. | Preparation example 3 | Preparation example 14 |
| Ex. 2 | (coating liquid D) | |

*Coating liquid D (Preparation example 3): a coating liquid containing a water-insoluble resin, a coating liquid containing a polyvinyl butyral resin (available from Sekisui Chemical Co., Ltd., BL-1, with a butyralization degree of 64 mol %) (5 parts by mass) and ethanol (95 parts by mass)

TABLE 2

| | | | Reaction 2 | | Color | |
|---|---|---|---|---|---|---|
| | | | (non-specific | | development | Color |
| | Reaction 1 (signal) | | adsorption) | | on surface of | development |
| | Line | | Line | | flow path | on surface of |
| | density | Judgment | density | Judgment | member | resin layer |
| Ex. 1 | 242 | A | No line | A | A | A |
| | | | detected | | | |
| Ex. 2 | 239 | A | No line | A | A | A |
| | | | detected | | | |
| Ex. 3 | 245 | A | No line | A | A | A |
| | | | detected | | | |
| Ex. 4 | 234 | A | No line | A | A | A |
| | | | detected | | | |
| Ex. 5 | 246 | A | No line | A | A | A |
| | | | detected | | | |
| Ex. 6 | 213 | B | No line | A | A | A |
| | | | detected | | | |
| Comp. Ex. 1 | 168 | C | 12 | B | B | A |
| Comp. Ex. 2 | 75 | D | No line | A | A | B |
| | | | detected | | | |

From the results of Table 1-1, Table 1-2, and Table 2, in Examples 1 to 6, a line that developed a color at a high density was obtained without detection of color development due to non-specific adsorption.

As compared, in Comparative Example 1, non-specific adsorption of the labeled antibody to the surface of the resin layer at a high density during an initial period after the testing liquid was spread was observed because there was no reagent deposited portion provided. Furthermore, the color developing intensity was low because there was no antibody bound with the surface of the flow path member.

In Comparative Example 2, the color developing intensity was low because no color development occurred on the surface of the resin layer (line) because there was no solid-phase reagent provided over the surface of the release and solid-phase reagent layer.

Aspects of the present disclosure are, for example, as follows:

<1> A testing device including:
a porous flow path member in which a testing liquid containing a sample is flowed;
a resin layer provided at at least one position over the flow path member;
a solid-phase reagent containing an antibody reactive with the sample; and
a reagent deposited portion that is water-soluble and contains a water-soluble resin and the solid-phase reagent,
wherein the resin layer contains a water-insoluble resin and is provided with the solid-phase reagent and the reagent deposited portion over a surface of the resin layer facing the flow path member, and
wherein the reagent deposited portion is disposed between the flow path member and the resin layer.
<2> The testing device according to <1>, including:
a porous flow path member in which a testing liquid containing a sample is flowed; and
a first resin layer and a second resin layer which are provided over the flow path member,
wherein the second resin layer is provided with a labeled antibody over a surface of the second resin layer facing the flow path member,
wherein the first resin layer contains a water-insoluble resin, and is provided with a capture antibody and a reagent deposited portion that is water-soluble and contains a water-soluble resin and the capture antibody, the capture antibody and the reagent deposited portion being provided over a surface of the first resin layer facing the flow path member, and wherein the reagent deposited portion is disposed between the flow path member and the first resin layer.
<3> The testing device according to <1> or <2>,
wherein the reagent deposited portion is eluted by the testing liquid.
<4> The testing device according to any one of <1> to <3>,
wherein the resin layer is not eluted by the testing liquid.
<5> The testing device according to <2> or <3>,
wherein the first resin layer is not eluted by the testing liquid.
<6> The testing device according to any one of <1> to <5>,
wherein an average thickness of the reagent deposited portion is 1 micrometer or greater but 20 micrometers or less.
<7> The testing device according to any one of <1> to <6>,
wherein the water-soluble resin in the reagent deposited portion is at least one selected from the group consisting of poly(ethylene oxide), polyethylene glycol, polyvinyl alcohol, polyvinyl pyrrolidone, polyacrylic acid, and acrylic resin.
<8> A testing kit including:
the testing device according to any one of <1> to <7>; and
at least one selected from the group consisting of a sample picking unit configured to pick a sample and a liquid for treating the sample.
<9> A transfer medium for producing a testing device, the transfer medium including:
a support;
a release layer provided over the support; and
a solid-phase reagent layer provided over the release layer,
wherein a reagent reactive with a sample and a reagent deposited portion are provided over a surface of the solid-phase reagent layer.
<10> A method for producing the testing device according to any one of <1> to <7>, the method including
a step of bringing the solid-phase reagent layer of the transfer medium for producing a testing device according to <9> into contact with a porous flow path member to transfer the solid-phase reagent layer onto the flow path member.
<11> A transfer medium for producing a testing device, the transfer medium including:
a support; and
a release and solid-phase reagent layer provided over the support,
wherein a reagent reactive with a sample and a reagent deposited portion are provided over a surface of the release and solid-phase reagent layer.
<12> A method for producing the testing device according to any one of <1> to <7>, the method including:
a step of bringing the release and solid-phase reagent layer of the transfer medium for producing a testing device according to <11> into contact with a porous flow path member to transfer the release and solid-phase reagent layer onto the flow path member.

The testing device according to any one of <1> to <7>, the testing kit according to <8>, the transfer medium for producing a testing device according to <9> or <11>, and the method for producing a testing device according to <10> or <12> can solve the various problems in the related art and can achieve the object of the present disclosure.

What is claimed is:

1. A testing device comprising:
a porous flow path member in which a testing liquid that comprises a sample is flowed;
a resin layer provided at at least one position over the flow path member;
a solid-phase reagent that comprises an antibody reactive with the sample; and
a reagent deposited portion that is water-soluble and comprises a water-soluble resin and the solid-phase reagent,
wherein the resin layer comprises a water-insoluble resin and is provided with the solid-phase reagent and the reagent deposited portion over a surface of the resin layer facing the flow path member, and
wherein the reagent deposited portion is disposed between the flow path member and the resin layer.

2. The testing device according to claim 1, comprising:
a porous flow path member in which a testing liquid that comprises a sample is flowed; and
a first resin layer and a second resin layer which are provided over the flow path member,
wherein the second resin layer is provided with a labeled antibody over a surface of the second resin layer facing the flow path member,
wherein the first resin layer comprises a water-insoluble resin, and is provided with a capture antibody and a reagent deposited portion that is water-soluble and comprises a water-soluble resin and the capture antibody, the capture antibody and the reagent deposited portion being provided over a surface of the first resin layer facing the flow path member, and
wherein the reagent deposited portion is disposed between the flow path member and the first resin layer.

3. The testing device according to claim 1,
wherein the reagent deposited portion is eluted by the testing liquid.

4. The testing device according to claim 1,
wherein the resin layer is not eluted by the testing liquid.

5. The testing device according to claim 2,
wherein the first resin layer is not eluted by the testing liquid.

6. The testing device according to claim 1,
wherein the flow path member comprises a hydrophilic porous material.

7. The testing device according to claim 6,
wherein the hydrophilic porous material is provided with one or more cells inside the hydrophilic porous material.

8. The testing device according to claim 7,
wherein the cell comprises a continuous cell.

9. The testing device according to claim 6,
wherein a voidage of the hydrophilic porous material is 40% or higher but 90% or lower.

10. The testing device according to claim 6,
wherein an average thickness of the hydrophilic porous material is 0.01 mm or greater but 0.3 mm or less.

11. The testing device according to claim 1,
wherein a content of the solid-phase reagent in the reagent deposited portion is 50% by mass or greater.

12. The testing device according to claim 1,
wherein an average thickness of the reagent deposited portion is 1 micrometer or greater but 20 micrometers or less.

13. A testing kit comprising:
the testing device according to claim 1; and
at least one selected from the group consisting of a sample picking unit configured to pick a sample and a liquid for treating the sample.

14. A transfer medium for producing a testing device, the transfer medium comprising:
a support;
a release layer provided over the support; and
a solid-phase reagent layer provided over the release layer,
wherein a reagent reactive with a sample and a reagent deposited portion are provided over a surface of the solid-phase reagent layer.

15. A method for producing a testing device, wherein the testing device comprises: a porous flow path member in which a testing liquid that comprises a sample is flowed; a resin layer provided at at least one position over the flow path member; a solid-phase reagent that comprises an antibody reactive with the sample; and a reagent deposited portion that is water-soluble and comprises a water-soluble resin and the solid-phase reagent, wherein the resin layer comprises a water-insoluble resin and is provided with the solid-phase reagent and the reagent deposited portion over a surface of the resin layer facing the flow path member, and
wherein the reagent deposited portion is disposed between the flow path member and the resin layer, the method comprising
bringing the solid-phase reagent layer of the transfer medium for producing a testing device according to claim 14 into contact with a porous flow path member to transfer the solid-phase reagent layer onto the flow path member.

16. The method for producing a testing device according to claim 15, wherein the reagent deposited portion is eluted by the testing liquid.

17. The method for producing a testing device according to claim 15, wherein the resin layer is not eluted by the testing liquid.

18. The method for producing a testing device according to claim 15, wherein the flow path member comprises a hydrophilic porous material.

19. A transfer medium for producing a testing device, the transfer medium comprising:
a support; and
a release and solid-phase reagent layer provided over the support, wherein a reagent reactive with a sample and a reagent deposited portion are provided over a surface of the release and solid-phase reagent layer.

20. A method for producing a testing device, wherein the testing device comprises: a porous flow path member in which a testing liquid that comprises a sample is flowed; a resin layer provided at at least one position over the flow path member; a solid-phase reagent that comprises an antibody reactive with the sample; and a reagent deposited portion that is water-soluble and comprises a water-soluble resin and the solid-phase reagent, wherein the resin layer comprises a water-insoluble resin and is provided with the solid-phase reagent and the reagent deposited portion over a surface of the resin layer facing the flow path member, and
wherein the reagent deposited portion is disposed between the flow path member and the resin layer, the method comprising
bringing the release and solid-phase reagent layer of the transfer medium for producing a testing device according to claim 19 into contact with a porous flow path member to transfer the release and solid-phase reagent layer onto the flow path member.
